SEQUENCE LISTING (24 sequences)

SEQ ID NO: 1 - protein, 1163 aa, Rattus norvegicus
MEDIDQSSLVSSSTDSPPRPPPAFKYQFVTEPEDEEDEEEEEDEEEDDEDLEELEVLERKPAAGLSAAAVPPAAAAPLLDFSSDSVPPAPRGPLPAAPPAAPERQPSWERSPAAPAPSLPPAAAVLPSKLPEDDEPPARPPPPPPAGASPLAEPAAPPSTPAAPKRRGSGSVDETLFALPAASEPVIPSSAEKIMDLMEQPGNTVSSGQEDFPSVLLETAASLPSLSPLSTVSFKEHGYLGNLSAVSSSEGTIEETLNEASKELPERATNPFVNRDLAEFSELEYSEMGSSFKGSPKGESAILVENTKEEVIVRSKDKEDLVCSAALHSPQESPVGKEDRVVSPEKTMDIFNEMQMSVVAPVREEYADFKPFEQAWEVKDTYEGSRDVLAARANVESKVDRKCLEDSLEQKSLGKDSEGRNEDASFPSTPEPVKDSSRAYITCASFTSATESTTANTFPLLEDHTSENKTDEKKIEERKAQIITEKTSPKTSNPFLVAVQDSEADYVTTDTLSKVTEAAVSNMPEGLTPDLVQEACESELNEATGTKIAYETKVDLVQTSEAIQESLYPTAQLCPSFEEAEATPSPVLPDIVMEAPLNSLLPSAGASVVQPSVSPLEAPPPVSYDSIKLEPENPPPYEEAMNVALKALGTKEGIKEPESFNAAVQETEAPYISIACDLIKETKLSTEPSPDFSNYSEIAKFEKSVPEHAELVEDSSPESEPVDLFSDDSIPEVPQTQEEAVMLMKESLTEVSETVAQHKEERLSASPQELGKPYLESFQPNLHSTKDAASNDIPTLTKKEKISLQMEEFNTAIYSNDDLLSSKEDKIKESETFSDSSPIEIIDEFPTFVSAKDDSPKLAKEYTDLEVSDKSEIANIQSGADSLPCLELPCDLSFKNIYPKDEVHVSDEFSENRSSVSKASISPSNVSALEPQTEMGSIVKSKSLTKEAEKKLPSDTEKEDRSLSAVLSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNSTIKELRRLFLVDDLVDSLKFAVLMWVFTYVGALFNGLTLLILALISLFSIPVIYERHQVQIDHYLGLANKSVKDAMAKIQAKIPGLKRKAD

SEQ ID NO: 2 - protein, 1192 aa, Homo sapiens
MEDLDQSPLVSSSDSPPRPQPAFRYQFVREPEDEEEEEEEEEEDEDEDLEELEVLERKPAAGLSAAPVPTAPAAGAPLMDFGNEFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMELKEQPGNTISAGQEDFPSVLLETAASLPSLSPLSAASFKEHEYLENLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKNSESSNDDTSFPSTPEGIKDRPGAYITCAPFNPAATESIATNIFPLLGDPTSENKTDEKKIEEKKAQIVTEKNTSTKTSNPFLVAAQESETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVQYESIKHEPENPPPYEEAMSVSLKKVSGIKEEIKEPENINAALQETEAPYISIACDLIKETKLSAEPAPEFSDYSEMAKVEQPVPDHSELVEDSSPDSEPVDLFSDDSIPDVPQKQDETVMLVKESLTETSFESMIEYEQKEKLSALPPEGGKPYLESFKLSLDNTKDTLLPDEVSTLSKKEKIPIQMEELSTAVYSNDDLFISKEAQIRETETFSDSSPIEIIDEFPTLISSKTDSFSKLAREYTDLEVSHKSEIAQAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 3 - DNA, 34 bp, Artificial Sequence
  Description of Artificial Sequence Synthetic primer
gctcagcggccgagaccctttttgctcttcctsg

SEQ ID NO: 4 - DNA, 28 bp, Artificial Sequence
  Description of Artificial Sequence Synthetic primer
gcttttaactatgctgcccatttctgst

SEQ ID NO: 5 - DNA, 40 bp, Artificial Sequence
  Description of Artificial Sequence Synthetic primer
ggtatccatgttctttaaaagaggcctgcgctacggtagc

SEQ ID NO: 6 - DNA, 63 bp, Artificial Sequence
  Description of Artificial Sequence Synthetic primer
cacttcacaggtcaagcttattaatggtgatggtgatggtgagcgcttttaactatgctgccc

SEQ ID NO: 7 - DNA, 43 bp, Artificial Sequence
  Description of Artificial Sequence Synthetic primer
ggtatccatgttctttaaaagaggcgccctgcgctacggtagc

SEQ ID NO: 8 - DNA, 38 bp, Artificial Sequence
  Description of Artificial Sequence Synthetic primer
gacattgagctcacccagtctccagcaatcatgkctgc

SEQ ID NO: 9 - DNA, 66 bp, Artificial Sequence
  Description of Artificial Sequence Synthetic primer
  modified_base (38)..(39): a, c, g, t, unknown or other
  modified_base (44)..(45): a, c, g, t, unknown or other
  modified_base (47)..(48): a, c, g, t, unknown or other
gcgcttcagctcgagcttggtcccagctccgaacgtmnnaggmnnmnntaacacattttgacagta

SEQ ID NO: 10 - DNA, 75 bp, Artificial Sequence
  Description of Artificial Sequence Synthetic primer
  modified_base (50)..(51): a, c, g, t, unknown or other
  modified_base (53)..(54): a, c, g, t, unknown or other
gcgcttcagctcgagcttggtcccagctccgaacgtaaccggcacccgmnnmnnattttgacagtaatacgttgc

SEQ ID NO: 11 - protein, 121 aa, Mus musculus
EVKLHESGPGLVRPGTSVKISCKASGYTFTNYWLGWVKQRPGHGLEWIGDIYPGGGYTNYNEKFKGKATLTADTSSSTAYMQLSSLTSEDSAVYFCARFYYGSSYWYFDVWGQGTTVTVSS

SEQ ID NO: 12 - protein, 107 aa, Artificial Sequence
  Description of Artificial Sequence Synthetic protein sequence
DIELTQSPAIMAASVGETVTITCGASENIYGALNWYQRKQGKSPQLLIYGATNLADGMSSRFSGSGSGRQYSLKISSLHPDDVATYYCQNINRVPVTFGAGTKLEIK

SEQ ID NO: 13 - DNA, 2248 bp, Artificial Sequence
  Description of Artificial Sequence Synthetic nucleotide sequence
  CDS (22)..(2238)
  sig_peptide (22)..(84)
  mat_peptide (85)..(2238)
tctagataacgagggcaaaaaatgaaaaagacagctatcgcgattgcagtggcactggctggtttcgctaccgtagcgcaggcctcttttaaagaacatggataccttggtaacttatcagcagtgtcatcctcagaaggaacaattgaagaaactttaaatgaagcttctaaagagttgccagagagggcaacaaatccatttgtaaatagagatttagcagaattttcagaattagaatattcagaaatgggatcatcttttaaaggctccccaaaaggagagtcagccatattagtagaaaacactaaggaagaagtaattgtgaggagtaaagacaaagaggatttagtttgtagtgcagcccttcacagtccacaagaatcacctgtgggtaaagaagacagagttgtgtctccagaaaagacaatggacatttttaatgaaatgcagatgtcagtagtagcacctgtgagggaagagtatgcagactttaagccatttgaacaagcatgggaagtgaaagatacttatgagggaagtagggatgtgctggctgctagagctaatgtggaaagtaaagtggacagaaaatgcttggaagatagcctggagcaaaaaagtcttgggaaggatagtgaaggcagaaatgaggatgcttctttccccagtaccccagaacctgtgaaggacagctccagagcatatattacctgtgcttcctttacctcagcaaccgaaagcaccacagcaaacactttccctttgttagaagatcatacttcagaaaataaaacagatgaaaaaaaaatagaagaaaggaaggcccaaattataacagagaagactagccccaaaacgtcaaatcctttccttgtagcagtacaggattctgaggcagattatgttacaacagataccttatcaaaggtgactgaggcagcagtgtcaaacatgcctgaaggtctgacgccagatttagttcaggaagcatgtgaaagtgaactgaatgaagccacaggtacaaagattgcttatgaaacaaaagtggacttggtccaaacatcagaagctatacaagaatcactttaccccacagcacagctttgcccatcatttgaggaagctgaagcaactccgtcaccagttttgcctgatattgttatggaagcaccattaaattctctccttccaagcgctggtgcttctgtagtgcagcccagtgtatccccactggaagcacctcctccagttagttatgacagtataaagcttgagcctgaaaaccccccaccatatgaagaagccatgaatgtagcactaaaagctttgggaacaaaggaaggaataaaagagcctgaaagttttaatgcagctgttcaggaaacagaagctccttatatatccattgcgtgtgatttaattaaagaaacaaagctctccactgagccaagtccagatttctctaattattcagaaatagcaaaattcgagaagtcggtgcccgaacacgctgagctagtggaggattcctcacctgaatctgaaccagttgacttatttagtgatgattcgattcctgaagtcccacaaacacaagaggaggctgtgatgctcatgaaggagagtctcactgaagtgtctgagacagtagcccagcacaaagaggagagacttagtgcctcacctcaggagctaggaaagccatatttagagtcttttcagcccaatttacatagtacaaaagatgctgcatctaatgacattccaacattgaccaaaaaggagaaaatttctttgcaaatggaagagtttaatactgcaatttattcaaatgatgacttactttcttctaaggaagacaaaataaaagaaagtgaaacattttcagattcatctccgattgagataatagatgaatttcccacgtttgtcagtgctaaagatgattctcctaaattagccaaggagtacactgatctagaagtatccgacaaaagtgaaattgctaatatccaaagcggggcagattcattgccttgcttagaattgccctgtgacctttctttcaagaatatatatcctaaagatgaagtacatgtttcagatgaattctccgaaaataggtccagtgtatctaaggcatccatatcgccttcaaatgtctctgctttggaacctcagacagaaatgggcagcatagttaaaagcgcttggcgtcacccgcagttcggtggttaataagctt

SEQ ID NO: 14 - DNA, 2425 bp, Artificial Sequence
  Description of Artificial Sequence Synthetic nucleotide sequence
  CDS (22)..(2415)
  sig_peptide (22)..(84)
  mat_peptide (85)..(2415)
tctagataacgagggcaaaaaatgaaaaagacagctatcgcgattgcagtggcactggctggtttcgctaccgtagcgcaggccgagaccctttttgctcttcctgctgcatctgagcctgtgataccctcctctgcagaaaaaattatggatttgatggagcagccaggtaacactgtttcgtctggtcaagaggatttcccatctgtcctgcttgaaactgctgcctctcttccttctctatctcctctctcaactgtttcttttaaagaacatggataccttggtaacttatcagcagtgtcatcctcagaaggaacaattgaagaaactttaaatgaagcttctaaagagttgccagagagggcaacaaatccatttgtaaatagagatttagcagaattttcagaattagaatattcagaaatgggatcatcttttaaaggctccccaaaaggagagtcagccatattagtagaaaacactaaggaagaagtaattgtgaggagtaaagacaaagaggatttagtttgtagtgcagcccttcacagtccacaagaatcacctgtgggtaaagaagacagagttgtgtctccagaaaagacaatggacatttttaatgaaatgcagatgtcagtagtagcacctgtgagggaagagtatgcagactttaagccatttgaacaagcatgggaagtgaaagatacttatgagggaagtagggatgtgctggctgctagagctaatgtggaaagtaaagtggacagaaaatgcttggaagatagcctggagcaaaaaagtcttgggaaggatagtgaaggcagaaatgaggatgcttctttccccagtaccccagaacctgtgaaggacagctccagagcatatattacctgtgcttcctttacctcagcaaccgaaagcaccacagcaaacactttccctttgttagaagatcatacttcagaaaataaaacagatgaaaaaaaaatagaagaaaggaaggcccaaattataacagagaagactagccccaaaacgtcaaatcctttccttgtagcagtacaggattctgaggcagattatgttacaacagataccttatcaaaggtgactgaggcagcagtgtcaaacatgcctgaaggtctgacgccagatttagttcaggaagcatgtgaaagtgaactgaatgaagccacaggtacaaagattgcttatgaaacaaaagtggacttggtccaaacatcagaagctatacaagaatcactttaccccacagcacagctttgcccatcatttgaggaagctgaagcaactccgtcaccagttttgcctgatattgttatggaagcaccattaaattctctccttccaagcgctggtgcttctgtagtgcagcccagtgtatccccactggaagcacctcctccagttagttatgacagtataaagcttgagcctgaaaaccccccaccatatgaagaagccatgaatgtagcactaaaagctttgggaacaaaggaaggaataaaagagcctgaaagttttaatgcagctgttcaggaaacagaagctccttatatatccattgcgtgtgatttaattaaagaaacaaagctctccactgagccaagtccagatttctctaattattcagaaatagcaaaattcgagaagtcggtgcccgaacacgctgagctagtggaggattcctcacctgaatctgaaccagttgacttatttagtgatgattcgattcctgaagtcccacaaacacaagaggaggctgtgatgctcatgaaggagagtctcactgaagtgtctgagacagtagcccagcacaaagaggagagacttagtgcctcacctcaggagctaggaaagccatatttagagtcttttcagcccaatttacatagtacaaaagatgctgcatctaatgacattccaacattgaccaaaaaggagaaaatttctttgcaaatggaagagtttaatactgcaatttattcaaatgatgacttactttcttctaaggaagacaaaataaaagaaagtgaaacattttcagattcatctccgattgagataatagatgaatttcccacgtttgtcagtgctaaagatgattctcctaaattagccaaggagtacactgatctagaagtatccgacaaaagtgaaattgctaatatccaaagcggggcagattcattgccttgcttagaattgccctgtgacctttctttcaagaatatatatcctaaagatgaagtacatgtttcagatgaattctccgaaaataggtccagtgtatctaaggcatccatatcgccttcaaatgtctctgctttggaacctcagacagaaatgggcagcatagttaaaagcgcttggcgtcacccgcagttcggtggttaataagctt

SEQ ID NO: 15 - DNA, 2278 bp, Artificial Sequence
  Description of Artificial Sequence Synthetic nucleotide sequence
  CDS (22)..(2268)
  sig_peptide (22)..(84)
  mat_peptide (85)..(2268)
tctagataacgagggcaaaaaatgaaaaagacagctatcgcgattgcagtggcactggctggtttcgctaccgtagcgcaggccgctagctggagccacccgcagttcgaaaaaggcgcctcttttaaagaacatggataccttggtaacttatcagcagtgtcatcctcagaaggaacaattgaagaaactttaaatgaagcttctaaagagttgccagagagggcaacaaatccatttgtaaatagagatttagcagaattttcagaattagaatattcagaaatgggatcatcttttaaaggctccccaaaaggagagtcagccatattagtagaaaacactaaggaagaagtaattgtgaggagtaaagacaaagaggatttagtttgtagtgcagcccttcacagtccacaagaatcacctgtgggtaaagaagacagagttgtgtctccagaaaagacaatggacatttttaatgaaatgcagatgtcagtagtagcacctgtgagggaagagtatgcagactttaagccatttgaacaagcatgggaagtgaaagatacttatgagggaagtagggatgtgctggctgctagagctaatgtggaaagtaaagtggacagaaaatgcttggaagatagcctggagcaaaaaagtcttgggaaggatagtgaaggcagaaatgaggatgcttctttccccagtaccccagaacctgtgaaggacagctccagagcatatattacctgtgcttcctttacctcagcaaccgaaagcaccacagcaaacactttccctttgttagaagatcatacttcagaaaataaaacagatgaaaaaaaaatagaagaaaggaaggcccaaattataacagagaagactagccccaaaacgtcaaatcctttccttgtagcagtacaggattctgaggcagattatgttacaacagataccttatcaaaggtgactgaggcagcagtgtcaaacatgcctgaaggtctgacgccagatttagttcaggaagcatgtgaaagtgaactgaatgaagccacaggtacaaagattgcttatgaaacaaaagtggacttggtccaaacatcagaagctatacaagaatcactttaccccacagcacagctttgcccatcatttgaggaagctgaagcaactccgtcaccagttttgcctgatattgttatggaagcaccattaaattctctccttccaagcgctggtgcttctgtagtgcagcccagtgtatccccactggaagcacctcctccagttagttatgacagtataaagcttgagcctgaaaaccccccaccatatgaagaagccatgaatgtagcactaaaagctttgggaacaaaggaaggaataaaagagcctgaaagttttaatgcagctgttcaggaaacagaagctccttatatatccattgcgtgtgatttaattaaagaaacaaagctctccactgagccaagtccagatttctctaattattcagaaatagcaaaattcgagaagtcggtgcccgaacacgctgagctagtggaggattcctcacctgaatctgaaccagttgacttatttagtgatgattcgattcctgaagtcccacaaacacaagaggaggctgtgatgctcatgaaggagagtctcactgaagtgtctgagacagtagcccagcacaaagaggagagacttagtgcctcacctcaggagctaggaaagccatatttagagtcttttcagcccaatttacatagtacaaaagatgctgcatctaatgacattccaacattgaccaaaaaggagaaaatttctttgcaaatggaagagtttaatactgcaatttattcaaatgatgacttactttcttctaaggaagacaaaataaaagaaagtgaaacattttcagattcatctccgattgagataatagatgaatttcccacgtttgtcagtgctaaagatgattctcctaaattagccaaggagtacactgatctagaagtatccgacaaaagtgaaattgctaatatccaaagcggggcagattcattgccttgcttagaattgccctgtgacctttctttcaagaatatatatcctaaagatgaagtacatgtttcagatgaattctccgaaaataggtccagtgtatctaaggcatccatatcgccttcaaatgtctctgctttggaacctcagacagaaatgggcagcatagttaaaagcgctcaccatcaccatcaccattaataagctt

SEQ ID NO: 16 - protein, 798 aa, Artificial Sequence
  Description of Artificial Sequence Synthetic protein sequence
MKKTAIAIAVALAGFATVAQAETLFALPAASEPVIPSSAEKIMDLMEQPGNTVSSGQEDFPSVLLETAASLPSLSPLSTVSFKEHGYLGNLSAVSSSEGTIEETLNEASKELPERATNPFVNRDLAEFSELEYSEMGSSFKGSPKGESAILVENTKEEVIVRSKDKEDLVCSAALHSPQESPVGKEDRVVSPEKTMDIFNEMQMSVVAPVREEYADFKPFEQAWEVKDTYEGSRDVLAARANVESKVDRKCLEDSLEQKSLGKDSEGRNEDASFPSTPEPVKDSSRAYITCASFTSATESTTANTFPLLEDHTSENKTDEKKIEERKAQIITEKTSPKTSNPFLVAVQDSEADYVTTDTLSKVTEAAVSNMPEGLTPDLVQEACESELNEATGTKIAYETKVDLVQTSEAIQESLYPTAQLCPSFEEAEATPSPVLPDIVMEAPLNSLLPSAGASVVQPSVSPLEAPPPVSYDSIKLEPENPPPYEEAMNVALKALGTKEGIKEPESFNAAVQETEAPYISIACDLIKETKLSTEPSPDFSNYSEIAKFEKSVPEHAELVEDSSPESEPVDLFSDDSIPEVPQTQEEAVMLMKESLTEVSETVAQHKEERLSASPQELGKPYLESFQPNLHSTKDAASNDIPTLTKKEKISLQMEEFNTAIYSNDDLLSSKEDKIKESETFSDSSPIEIIDEFPTFVSAKDDSPKLAKEYTDLEVSDKSEIANIQSGADSLPCLELPCDLSFKNIYPKDEVHVSDEFSENRSSVSKASISPSNVSALEPQTEMGSIVKSAWRHPQFGG

SEQ ID NO: 17 - protein, 739 aa, Artificial Sequence
  Description of Artificial Sequence Synthetic protein sequence
MKKTAIAIAVALAGFATVAQASFKEHGYLGNLSAVSSSEGTIEETLNEASKELPERATNPFVNRDLAEFSELEYSEMGSSFKGSPKGESAILVENTKEEVIVRSKDKEDLVCSAALHSPQESPVGKEDRVVSPEKTMDIFNEMQMSVVAPVREEYADFKPFEQAWEVKDTYEGSRDVLAARANVESKVDRKCLEDSLEQKSLGKDSEGRNEDASFPSTPEPVKDSSRAYITCASFTSATESTTANTFPLLEDHTSENKTDEKKIEERKAQIITEKTSPKTSNPFLVAVQDSEADYVTTDTLSKVTEAAVSNMPEGLTPDLVQEACESELNEATGTKIAYETKVDLVQTSEAIQESLYPTAQLCPSFEEAEATPSPVLPDIVMEAPLNSLLPSAGASVVQPSVSPLEAPPPVSYDSIKLEPENPPPYEEAMNVALKALGTKEGIKEPESFNAAVQETEAPYISIACDLIKETKLSTEPSPDFSNYSEIAKFEKSVPEHAELVEDSSPESEPVDLFSDDSIPEVPQTQEEAVMLMKESLTEVSETVAQHKEERLSASPQELGKPYLESFQPNLHSTKDAASNDIPTLTKKEKISLQMEEFNTAIYSNDDLLSSKEDKIKESETFSDSSPIEIIDEFPTFVSAKDDSPKLAKEYTDLEVSDKSEIANIQSGADSLPCLELPCDLSFKNIYPKDEVHVSDEFSENRSSVSKASISPSNVSALEPQTEMGSIVKSAWRHPQFGG

SEQ ID NO: 18 - protein, 749 aa, Artificial Sequence
  Description of Artificial Sequence Synthetic protein sequence
MKKTAIAIAVALAGFATVAQAASWSHPQFEKGASFKEHGYLGNLSAVSSSEGTIEETLNEASKELPERATNPFVNRDLAEFSELEYSEMGSSFKGSPKGESAILVENTKEEVIVRSKDKEDLVCSAALHSPQESPVGKEDRVVSPEKTMDIFNEMQMSVVAPVREEYADFKPFEQAWEVKDTYEGSRDVLAARANVESKVDRKCLEDSLEQKSLGKDSEGRNEDASFPSTPEPVKDSSRAYITCASFTSATESTTANTFPLLEDHTSENKTDEKKIEERKAQIITEKTSPKTSNPFLVAVQDSEADYVTTDTLSKVTEAAVSNMPEGLTPDLVQEACESELNEATGTKIAYETKVDLVQTSEAIQESLYPTAQLCPSFEEAEATPSPVLPDIVMEAPLNSLLPSAGASVVQPSVSPLEAPPPVSYDSIKLEPENPPPYEEAMNVALKALGTKEGIKEPESFNAAVQETEAPYISIACDLIKETKLSTEPSPDFSNYSEIAKFEKSVPEHAELVEDSSPESEPVDLFSDDSIPEVPQTQEEAVMLMKESLTEVSETVAQHKEERLSASPQELGKPYLESFQPNLHSTKDAASNDIPTLTKKEKISLQMEEFNTAIYSNDDLLSSKEDKIKESETFSDSSPIEIIDEFPTFVSAKDDSPKLAKEYTDLEVSDKSEIANIQSGADSLPCLELPCDLSFKNIYPKDEVHVSDEFSENRSSVSKASISPSNVSALEPQTEMGSIVKSAHHHHHH

SEQ ID NO: 19 - protein, 107 aa, Artificial Sequence
  Description of Artificial Sequence Synthetic protein sequence
DIELTQSPAIMAASVGETVTITCGASENIYGALNWYQRKQGKSPQLLIYGATNLADGMSSRFSGSGSGRQYSLKISSLHPDDVATYYCQNVLSTPRTFGAGTKLEIK

SEQ ID NO: 20 - protein, 107 aa, Artificial Sequence
  Description of Artificial Sequence Synthetic protein sequence
DIELTQSPAIMAASVGETVTITCGASENIYGFLNWYQRKQGKSPQLLIYGATNLADGMSSRFSGSGSGRQYSLKISSLHPDDVATYYCQNVLSTPRTFGAGTKLEIK

SEQ ID NO: 21 - protein, 107 aa, Artificial Sequence
  Description of Artificial Sequence Synthetic protein sequence
DIELTQSPAIMAASVGETVTITCGASENIYGALNWYQRKQGKSPQLLIYGATNLADGMSSRFSGSGSGRQYSLKISSLHPDDVATYYCQNVLRVPCTFGAGTKLEIK

SEQ ID NO: 22 - protein, 107 aa, Artificial Sequence
  Description of Artificial Sequence Synthetic protein sequence
DIELTQSPAIMAASVGETVTITCGASENIYGALNWYQRKQGKSPQLLIYGATNLADGMSSRFSGSGSGRQYSLKISSLHPDDVATYYCQNVLRVPVTFGAGTKLEIK

SEQ ID NO: 23 - protein, 5 aa, Artificial Sequence
  Description of Artificial Sequence Synthetic 5xHis tag
HHHHH

SEQ ID NO: 24 - protein, 6 aa, Artificial Sequence
  Description of Artificial Sequence Synthetic 6xHis tag
HHHHHH

(19) United States
(12) Patent Application Publication
(10) Pub. No.: US 2006/0287501 A1
(43) Pub. Date: Dec. 21, 2006

(54) Soluble truncated polypeptides of the nogo-a protein

(75) Inventors: Arne Skerra, Freising (DE); Markus Fiedler, Halle/Saale (DE)

Correspondence Address: FOLEY AND LARDNER LLP;SUITE 500, 3000 K STREET NW, WASHINGTON, DC, 20007, US

(73) Assignee: PIERIS PROTEOLAB AG, Freising-Weihenstephan (DE)

(21) Appl. No.: 10/533,299

(22) PCT Filed: Oct. 31, 2002
(86) PCT No.: PCT/EP02/12210

§ 371 Date: Feb. 13, 2006

(51) Int. Cl. G01N 33/567; C07H 21/04; C12P 21/04; C07K 14/705; C07K 16/28

(52) U.S. Cl. 530/350; 435/69.7; 435/320.1; 435/325; 536/23.5; 435/7.2; 530/388.22

(57) ABSTRACT

The present invention refers to an isolated truncated Nogo-A polypeptide that corresponds to a truncated form of the Nogo-A protein consisting of the amino acids 174 to 940 of the full length protein of rat Nogo-A or of the amino acids 246 to 966 of the human full length Nogo-A protein.

The present invention relates to soluble truncated polypeptides of the Nogo-A protein, nucleic acid molecules encoding such polypeptides as well as to methods for the production of such polypeptides. The present invention also relates to methods for identifying and generating compounds having detectable affinity to a Nogo-A protein, in particular such compounds that have a neutralizing effect on the neurite-growth-inhibiting activity of Nogo-A. Therefore, the present invention is also directed to the use of compounds having binding affinity and preferably also a neutralizing effect on the neurite-growth-inhibiting activity of Nogo-A as diagnostics or pharmaceuticals.

The very limited capacity of the adult central nervous system (CNS) for axonal regeneration is a phenomenon of broad and ongoing scientific as well as medical interest (see, e.g., Horner and Gage, (2000) Nature, 407, 963-970). In contrast, sprouting and elongation of lesioned axons readily occurs in the peripheral nervous system (PNS). Inhibitory effects and non-permissible properties of CNS tissue, in particular of CNS myelin and oligodendrocytes, probably contribute considerably to the restriction of neuronal regeneration and plasticity. In vitro, CNS myelin and oligodendrocyte membranes induce growth cone collapse (Bandtlow et al., (1990) J. Neurosci., 10, 3837-3848).

Based on earlier observations of the inhibitory effect of CNS myelin on neurite outgrowth (Caroni and Schwab, J. Cell Biol., (1988) 106, 1281-1288) the myelin-associated neurite growth inhibitor NI-220 (Spilmann et al., (1998) J. Biol. Chem., 273, 19283-1929), later called Nogo-A (Huber and Schwab, Biol. Chem., 381, 407-419), was identified in bovine spinal cord tissue as a predominant protein of oligodendrocytes that prevents axonal growth. The corresponding cDNAs from rat and man were recently described (Chen et al., (2000) Nature, 403, 434-439; GrandPré, et al., (2000) Nature, 403, 439-444; Prinjha et al., (2000) Nature, 403, 383-384). The nogo gene encodes three distinct proteins, Nogo-A, Nogo-B, and Nogo-C, which apparently arise by alternative splicing and/or promoter usage. Of those only the full length Nogo-A transcript is specifically expressed in oligodendrocytes and hence made mainly responsible for their neuronal growth inhibitory activity (Spillmann et al., supra; Chen et al., supra).

In addition, a monoclonal antibody named IN-1 is known (Caroni and Schwab, (1988) Neuron, 1, 85-96; European Patent Application 0 396 719). This antibody was shown to neutralize the inhibitory activity of Nogo in vitro (Bandtlow et al., (1990) J. Neurosci., 10, 3837-3848; Spillmann et al., supra) and in vivo, giving rise to long-distance regeneration and improved plastic changes of injured CNS fiber tracts (Schnell and Schwab, (1990) Nature, 343, 269-272; Z'Graggen et al., (1998) J. Neurosci., 18, 4744-4757).

The variable domain cDNAs of the antibody IN-1 were cloned from the hybridoma cell line, followed by the bacterial production of the corresponding recombinant murine F_{ab} fragment, whose functionality was demonstrated in vitro (Bandtlow et al., (1996) Eur. J. Biochem., 241, 468-475). A partially humanized IN-1 F_{ab} fragment was produced by E. coli fermentation and shown to successfully promote regeneration of corticospinal axons in adult rats after spinal cord lesion in vivo (Broesamle et al., (2000) J. Neurosci., 20, 8061-8068). The recombinant IN-1 F_{ab} fragment also induced significant elongation of injured cochlear fibres upon intrathecal treatment (Tatagiba et al., (2002) Acta Neurochir. (Wien), 144, 181-187) and a pronounced sprouting response of Purkinje cells after injection into the intact adult cerebellum (Buffo et al.,(2000) J. Neurosci., 20, 2275-2286).

However, two problems exist for studying axonal growth and for developing methods for promoting neuronal regeneration in the CNS.

First, as a membrane-bound protein Nogo-A is traditionally isolated only in small amounts and in a laborious procedure from CNS myelin. The heterologous production of the full length 1163 Nogo-A protein (1163 residues in case of the rat Nogo-A, 1192 residues in case of the human protein) in mammalian cells (Chen et al., supra; GrandPré, et al., supra, is apparently also not suitable for providing the rather large amounts of pure protein which are, for example, needed to study the inhibitory activity of Nogo at the molecular level (e.g. by X-ray crystallography) or in screening assays for compounds with neutralizing activity. According to Chen et al., supra enrichment of recombinant Nogo by means of affinity chromatography yielded a protein extract from CHO cells in which Nogo represented only about 1 to 5% of the protein present.

In addition, Prinjha et al., supra describe the production of a soluble fusion protein of human Nogo-A in which amino acid residues 1 to 1024 are fused to a human Fc polypeptide. Furthermore, GrandPré et al., supra, describe the expression of a 66-residue lumenal/extracellular fragment of human Nogo (amino acids 1055 to 1120 of human Nogo-A) as fusion protein with glutathione S-transferase (GST). Both fusion proteins are reported to be a potent neurite-growth inhibitor. However, no further use of these fusion proteins in investigating the inhibitory effect of Nogo or in the development of potential pharmacological treatments have been described.

Second, the only molecule for which a notable neutralizing effect on the neurite-growth-inhibiting activity of Nogo-A has been observed is the antibody IN-1. However, both the original monoclonal antibody IN-1 as well as its bacterially produced F_{ab} fragment have a rather low affinity for the antigen Nogo-A. Due to this low affinity, and in case of the monoclonal IgM antibody also due to its large size, the antibody IN-1 do not represent a well-suited candidate for practical applications, in particular for therapeutic purposes.

Therefore, there is still a demand for an assay system with which, a) regeneration processes can be investigated at the molecular level, and b) molecules having improved binding affinity to Nogo-A, and optionally also with improved neutralizing effect on the neurite-growth-inhibiting activity of Nogo-A, can be found.

Accordingly, it is an object of the invention to overcome the limitations of the prior art and to provide a system that meets the above needs.

This object is solved, among others, by the polypeptides and the method having the features of the independent claims.

Such a polypeptide is an isolated truncated Nogo-A polypeptide that corresponds to a truncated form of the Nogo-A protein consisting of the amino acids 174 to 940 of the full length protein of rat Nogo-A (SEQ ID NO: 1, 1163 amino acids) or of the amino acids 246 to 966 of the human full length protein (SEQ ID NO: 2, 1192 amino acids).

The inventors have found that such a N— and C-terminally truncated form of the Nogo-A protein has many advantages. First, it can be produced as a soluble, stable protein,that does not undergo significant proteolytic degradation, without using a fusion protein that confers solubility. Second, this polypeptide can be produced in amounts that are sufficient, for example, for large scale screening assays or crystallization experiments. Third, the truncated soluble protein maintains the neurite-growth-inhibiting activity of the full length protein. This is in so far surprising as the so-called “Nogo-66” region comprising the amino acid residues 1055 to 1120 of human Nogo-A, that belong to that C-terminal part of the full length protein that is deleted in the fragments of the present invention, was recently reported to be a potent nerve cone collapsing factor, i.e. a potent inhibitor of the axonal regeneration (GrandPré, et al., supra). Consequently, the good stability and availability of the inventive truncated Nogo-A protein together with its inhibitory activity render it to be an excellent target that can be used in the screening for molecules having neutralizing activity.

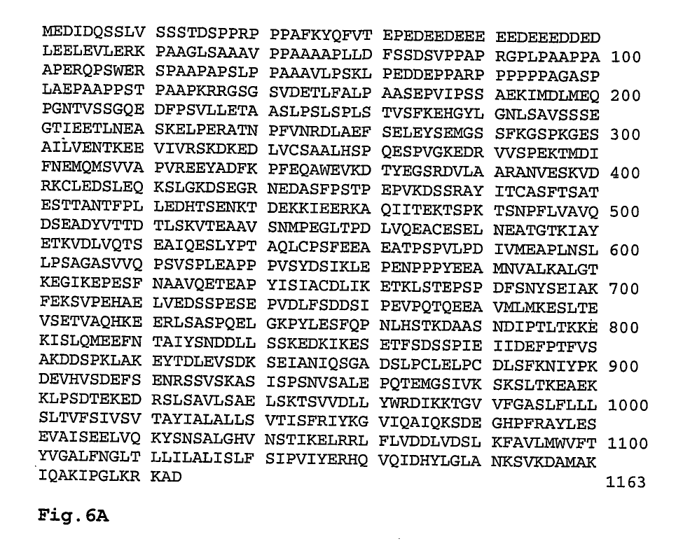

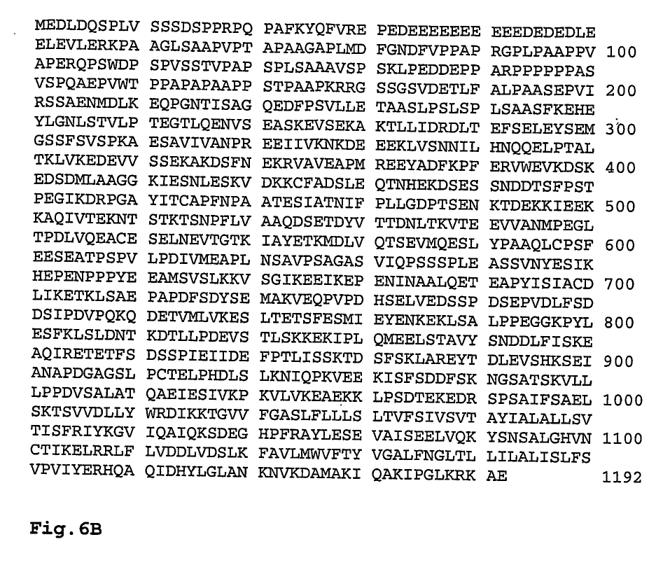

For reasons of clarity it is noted that the numbering of the amino acid residues, when referring to the rat protein, is used in accordance with the numbering of the 1163 residues containing full length protein of rat described by Chen et al, supra (SEQ ID NO: 1, EMBL data base accession code: AJ242961). When referring to the human protein, the residue numbering is used in accordance with the sequence of the full length human protein (SEQ ID NO:2, EMBL data base accession number AJ251383; 1192 residues) described by GrandPré, et al., supra and Prinjha et al., supra, (cf. FIG. 6 where the amino acid sequences as deposited as also shown). It is noted in this respect, that the present results indicate that the truncated fragments of Nogo-A according to the present invention are derived from one exon of the gene.

In a preferred embodiment, the polypeptide of the invention corresponds to the truncated form of the Nogo-A protein which consists of the amino acids 223 to 940 of the full length protein of rat Nogo-A. In a further embodiment, this truncated polypeptide corresponds to the Nogo-A protein that consists of the amino acids 270 to 900 of the full length protein of rat Nogo-A. Generally speaking, a preferred truncated polypeptide of the invention corresponds to a truncated Nogo-A protein of rat that comprises at least the sequences positions 323 to 890 in order to be able to include all cysteine residues that are present at positions 323, 403, 443, 536, 676, 885 and 890 in the wild-type rat protein.

In a further preferred embodiment, the polypeptide corresponds to a truncated form of the Nogo-A protein that consists of the amino acids 334 to 966 of the full length human Nogo-A protein. Preferably, the truncated form of the Nogo-A protein consists of the amino acids 380 or 424 to 699 or 850 of the full length human Nogo-A protein. In an alternative embodiment, the truncated Nogo-A polypeptide corresponds to a truncated human Nogo-A protein that comprises at least the sequences positions 424, 464, 559, 596, 699 and 912 which are occupied by cysteine residues in the human wild-type protein.

In general the truncated Nogo-A protein is not limited to a specific lower size but every truncated form falling within the boundaries defined by the amino acid positions 174 to 940 of the full length protein of rat Nogo-A (SEQ ID NO: 1, 1163 amino acids) or 246 to 966 of the human full length protein, respectively, are in the scope of the invention as long as they have similar or the same inhibitory activity as the respective Nogo-A wild type protein and/or preferably fold into a polypeptide having a three-dimensional structure similar or identical to the wild type protein. Accordingly, truncated Nogo-A forms having a length of (only) e.g. 19, 20, 25, 50, 100, 150 or 200, 250 or 300 residues are also comprised in the invention if they yield a functional active Nogo-A peptide or protein. The functionality can be assessed in a common neurite outgrowth assay as described here or e.g. by Chen et al., supra, or by GrandPré et al., supra. In one aspect, fragments are preferred which include all cysteine residues that seem to play a role in the folding of the protein. In case of the Nogo-A protein of rat, such a fragment includes the sequence corresponding to positions 323 to 890 of the full length Nogo-A sequence. In the case of the human protein, such a fragment includes the amino acid residues 424 to 699 or 424 to 890 (cf. above).

The truncated form of the Nogo-A protein of the invention can be derived from the natural sequence of any suitable mammal and non-mammal species. Although the truncated polypeptide is preferably of mammalian origin, for instance of human, porcine, murine, bovine or rat origin, the use of Nogo orthologues from invertebrates or lower species such as Drosophila melanogaster or Caenorhabditis elegans is also within the scope of the invention. In one preferred embodiment the mutein is a truncated variant of Nogo-A protein of human or rat origin.

In preferred embodiments the polypeptide of the present invention is selected from the group consisting of:

a) the polypeptide having the amino acid sequence consisting of amino acid residues 174 to 940 of the full length rat Nogo-A protein (SEQ ID NO: 1);

b) the polypeptide having the amino acid sequence consisting of amino acid residues 233 to 940 of the full length rat Nogo-A protein (SEQ ID NO: 1);

c) the polypeptide having the amino acid sequence consisting of amino acid residues 246 to 966 of the full length human Nogo-A protein (SEQ ID NO: 2);

d) the polypeptide having the amino acid sequence consisting of the amino acid residues 334 to 966 of the full length human Nogo-A protein (SEQ ID NO: 2);

e) a polypeptide having at least 50% sequence identity to any of the polypeptides a) to d) wherein the fragment of human Nogo-A consisting of amino acids 1 to 1024 is excluded;

f) a fragment of any of the polypeptides a) to e), wherein the fragment consisting of amino acids 624 to 639 of the full length rat Nogo-A protein is excluded.

As stated above, such a fragment of a truncated Nogo-A protein can contain not more than 19, 20, 50, 100, 150, 200, 250 or 300 amino acid residues.

The term “sequence identity” or “identity” as used in the present invention means the percentage of pair-wise identical residues—following homology alignment of a sequence of a polypeptide of the present invention with a sequence in question—with respect to the number of residues in the longer of these two sequences.

Thus taking, for example, as polypeptide in question the polypeptide that is used in Chen et al., supra for the generation of the antiserum “AS Bruna” and that consists of the amino acid residues 762 to 1163 (i.e. 402 residues) of the full length rat protein, the identity as defined in the present invention is calculated as follows. Compared to the fragment of the invention consisting of amino acids 174 to 940 of the rat Nogo-A, this “AS Bruna” polypeptide shares (following homology alignment) 940−762=179 pair-wise identical residues with the inventive polypeptide. Since the polypeptide of the invention is the longer of the two fragments (767 residues), the identity is calculated to be 179/767=0.233 or 23.3%. As a second example, the identity of this “AS bruna” polypeptide with a inventive fragment consisting of amino acid residues 233 to 890 of the rat full length Nogo-A is as follows. The “AS Bruna” polypeptide shares 890−762=129 identical residue with the polypeptide of the invention. Again the polypeptide of the invention is the longer fragment (890−233=658 residues). The identity is thus 129/658=0.196 or 19.6%.

In a further preferred embodiment the truncated human Nogo-A polypeptide of the invention begins with an amino acid residue selected from the amino acids 246 to 424 and ends at a residue selected from amino acids 912 to 966 of the full length protein. A preferred truncated polypeptide of the rat Nogo-A protein begins with an amino acid residue selected from the amino acids 174 to 233 and ends at a residue selected from amino acids 890 to 940 of the full length Nogo-A.

In accordance with the above definition of the term “identity”, the polypeptide of the invention can have the natural amino acid sequence of Nogo-A throughout the truncated form. On the other hand, the truncated polypeptide disclosed here can also contain amino acid mutations compared to the wild-type protein as long as those mutations a) do not yield a protein with less that 50% sequence identity and preferably b) yield a protein that folds into a three-dimensional structure identical or comparable to that of one of the truncated forms of Nogo-A of the present invention and/or has the same biological neurite growth inhibitory activity. This also means, that a polypeptide having a sequence identity of equal to or greater than 50% is also considered to fall within the scope of the present invention, even if it does not have any neurite growth inhibitory activity at all but a different biological activity.

The differences in the amino acid sequence can be caused, for example, by mutations, substitutions, deletions, insertion (of continuous stretches) of amino acid residues as well as by N— and/or C-terminal additions introduced into the natural amino acid sequence of the truncated Nogo-A forms, i.e. the truncated Nogo-A consisting of amino acid residues 174 to 940 of the full length rat Nogo-A protein (SEQ ID NO: 1) or amino acid residues 246 to 966 of the full length human Nogo-A protein (SEQ ID NO:2) or a smaller fragment thereof as disclosed herein.

Such modifications of the amino acid sequence within or outside these boundaries of the selected protein include directed mutagenesis of single amino acid positions, for example, in order to simplify the subcloning of the Nogo gene or its parts by incorporating cleavage sites for certain restriction enzymes. Furthermore, mutations can be introduced within the truncated polypeptide in order to improve certain characteristics of the chosen Nogo-A protein, for example its folding stability or folding efficiency or its resistance to proteases. For example, if recombinant production is to take place in an oxidizing thiol/disulfide redox milieu in vivo or if the protein is to be used in an oxidizing environment, cysteine residues can be replaced by serine or alanine in order to avoid processes such as dimerization or oxidation of the thiol group which deteriorate the folding efficiency or the life-time of the purified protein when stored. Therefore, the cysteine residues that are not crucial for the folding of the protein can be replaced in the Nogo-A variants of the present invention. In one embodiment of fragments which are based on Nogo-A of rat origin, at least one of the cysteine residues at positions 403, 536, 574 and 676 are substituted by a suitable amino acid (cf. Examples).

In preferred embodiments, the polypeptide of the invention has at least 60, 70, 72, 75, 80, 85, or 90 or 95% sequence identity to the truncated form of the Nogo-A protein described here. In accordance with the meaning of the term “identity”, the substitution of an amino acid with a chemically similar amino acid is considered to be a conservative substitution that maintains the identity. Examples of such conservative substitutions are the substitution for one another: 1) alanine, serine, threonine; 2) aspartic acid and glutamatic acid; 3) asparagine and glutamine; 4) arginine and lysine; 5) isoleucine, leucine, methionine, valin; and 6) phenylalanine, tyrosine, tryptophan.

Although the Nogo-A protein of the present invention comprises a stable soluble monomeric polypeptide chain which can produced as such, it is also possible to produce the truncated Nogo-A protein as fusion protein. The fusion partner can be connected to the N— and/or the C-terminus of the Nogo-A polypeptide and is preferably a protein, a protein 5 domain or a peptide. In case of a peptide, this peptide is preferably an affinity tag such as the Strep-Tag® or the Strep-tag® II (Schmidt et al., J. Mol. Biol. 255 (1996), 753-766) or an oligohistidine, e.g. penta- or hexahistidine tag.

For the heterologous production, a peptide such as a signal sequence and/or an affinity tag is operably fused to the N-terminus or to the C-terminus of the Nogo-A protein. Affinity tags such as the Strep-Tag® or the Strep-tag® II (Schmidt et al., supra) or oligohistidine tags (e.g., His₅- or His₆-tags) or proteins such as glutathione-S-transferase which can be used for purification by affinity chromatography and/or for detection (e.g. using the specific affinity of the Strep-tag® for streptavidin) are examples of preferred fusion partners. Further examples of fusion partners which can be advantageous in practice are binding domains such as the albumin-binding domain of protein G, the immunoglobulin-binding domains of protein A or oligomerizing domains, if, for example, an avidity effect is desired. As indicated, the term fusion protein as used herein also includes truncated Nogo-A polypeptides that are equipped with a signal sequence. Signal sequences at the N-terminus of a polypeptide according to the invention can be suitable to direct the polypeptide to a specific cell compartment during its biosynthesis, for example into the periplasm of E. coli or to the lumen of the endoplasmic reticulum of the eukaryotic cell or into the medium surrounding the cell. In doing so, the signal sequence is usually cleaved by a signal peptidase. It is also possible to use other targeting or signalling sequences which may also be located at the N-terminus of the polypeptide and which allow the localization thereof in specific cell compartments. A preferred signal sequence for secretion into the periplasm of E. coli is the OmpA signal sequence. A large number of further signal sequences is known in the art.

Therefore, the present invention is also directed to a method for the production of a truncated Nogo-A polypeptide or a fusion protein thereof. In this method, the Nogo-A polypeptide or the fusion protein of the Nogo-A polypeptide is produced starting from the nucleic acid coding for the Nogo-A polypeptide either by means of an in vitro transcription and translation system (e.g. a cell free system) or by means of genetic engineering methods either in in a bacterial or eukaryotic host organism. The polypeptide is then isolated from this in vitro system or from this host organism or its culture.

For this purpose a suitable host cell is usually first transformed with a vector comprising a nucleic acid molecule encoding, for instance, the truncated human Nogo-A consisting of amino acid residues 334 to 966 of the invention. The host cell, which can be any prokaryotic or eukaryotic host cell is then cultured under conditions which allow the biosynthesis of the polypeptide (via transcription/translation of the nucleic acid or gene). The polypeptide is then usually recovered either from the cell or from the cultivation medium. Since the Nogo-A protein seems to contain structural disulfide bonds it is preferred to direct the polypeptide into a cell compartment having an oxidizing thiol/disulfide redox milieu by use of a suitable signal sequence. Such an oxidizing milieu is present in the periplasm of bacteria such as E coli or in the lumen of the endoplasm reticulum of a eukaryotic cell and usually favours the correct formation of the disulfide bonds. It is, however, also possible to produce a polypeptide of the invention in the cytosol of a host cell preferably E coli. In this case the polypeptide can, for instance, be produced in form of inclusion bodies, followed by renaturation in vitro. A further option is the use of specifically mutated strains which have an oxidizing milieu in the cytosol and thus allow allow production of the native protein in the cytosol.

The invention is also related to a nucleic acid molecule encoding a truncated Nogo-A polypeptide according to the invention or a fusion protein thereof

In one preferred embodiment the nucleic acid molecule consists of or comprises the nucleotide sequence of positions 522 to 2822 of the coding sequence of rat Nogo-A (encoding the amino acids 174 to 940 of rat Nogo-A) deposited under accession number AJ242961 in the EMBL database or the nucleotide sequence of positions 699 to 2822 (encoding the amino acids 233 to 940 or rat Nogo-A) of this coding sequence. In another preferred embodiment the nucleic acid molecule consists of or comprises the nucleotide sequence of positions 738 to 2900 of the coding sequence of human Nogo-A (encoding the amino acids 246 to 966 or human Nogo-A) deposited under accession number AJ251383 in the EMBL data or of positions 1002 to 2900 of this coding sequence (encoding the amino acids 334 to 966 of human Nogo-A).

Since the degeneracy of the genetic code permits substitutions of certain codons by other codons which specify the same amino acid and hence give rise to the same protein, the invention is not limited to a specific nucleic acid molecule but includes all nucleic acid molecules comprising a nucleotide sequence coding for a truncated Nogo protein with an amino acid sequence according to the present invention.

The nucleic acid molecule encoding a truncated Nogo-A polypeptide disclosed here can be operably linked to a regulatory sequence to allow expression of the nucleic acid molecule in a host cell (in vivo) or its transcription and translation in a cell-free system (in vitro).

A nucleic acid molecule such a DNA is regarded to be “capable of expressing a polypeptide” if it contains nucleotide sequences which contain transcriptional and translational information and if such sequences are “operably linked” to nucleotide sequences which encode the polypeptide. An operable linkage is a linkage in which the regulatory DNA sequences and the DNA sequences sought to be expressed are connected in such a way as to permit gene expression. The precise nature of the regulatory regions and elements needed for gene expression may vary from organism to organism, but shall, in general, include a promoter region which, in prokaryotes for example, contains both the promoter regulatory sequence that can comprise a transcriptional region functional in a cell and a transcriptional terminating region functional in a cell. Elements used for transcription or translation are promoters, operators, enhancers, leader sequences, transcription initiation sites and transcription termination sites, polyadenylation signals, ribosomal binding sites such the Shine-Dalgarno sequence and the like. The gene expression may also be inducible. These regulatory sequences and/or the truncated Nogo-A protein of the invention can be part of a vector. Accordingly, the invention also refers to a vector comprising a nucleic acid sequence coding for the truncated Nogo-A protein as disclosed here.

In a further aspect, the present invention refers to a method for identifying a compound having detectable affinity to a Nogo-A protein, comprising the steps of:

(a) contacting a truncated Nogo-A polypeptide or a fusion protein thereof as defined above with a compound of interest under conditions that allow formation of a complex between the truncated Nogo-A protein and said compound; and

(b) detecting complex formation by means of a suitable signaling method.

In an alternative embodiment the method for identifying a compound having detectable affinity to a Nogo-A protein comprising the steps of:

(a) contacting a truncated Nogo-A polypeptide or a fusion protein thereof as defined above with a plurality of compounds of interest under conditions that allow formation of a complex between the truncated Nogo-A protein and said compounds; and

(b) enriching at least one compound of interest that has detectable binding affinity to the Nogo-A protein by screening or selection and/or isolating said at least one compound.

Thus, by use of the truncated Nogo-A proteins disclosed here, the invention provides for the first time a method which can be used in screening assays, e.g. using high throughput screening systems or evolutionary methods (combinatorial biology), for obtaining compounds having binding activity to a (wild-type) Nogo-A protein. For reason of clarity, it is noted that the term “a Nogo-A protein” is not restricted to a specific source but is to include Nogo-A proteins from mammalian and non-mammalian source, for example.

The term “plurality” as used herein means that at least two compounds that differ from each other in their structure, for example, in their amino acid or nucleotide sequences are present.

The method of identifying a compound having detectable affinity can be carried out with compounds (of interest) for which a binding affinity to Nogo-A has not been reported so far. However, the method of the invention can also be used for finding molecules starting from a (lead) compound which is known to bind a Nogo-A protein. Preferably the compound having detectable affinity is an organic molecule, a peptide, a polypeptide or a nucleic acid.

The term “organic molecule” preferably means an organic molecule comprising at least two carbon atoms, but not more than 7 rotatable carbon bonds having a molecular weight between 100 and 2000 Dalton, preferably 1000 Dalton and also a molecule including one or two metal atoms.

The signaling method used for detecting complex formation between the truncated Nogo-A protein and the binding compound may use every suitable signaling means which directly or indirectly generates in a chemical, enzymatic or physical reaction a detectable compound or a signal that can be used for detection. An example for a physical reaction is the emission of fluorescence after excitation with radiation or the emission of e.g. α- or β-radiation by a radioactive label; alkaline phosphatase, horseradish peroxidase or β-galactosidase are examples of enzyme labels which catalyse the formation of chromogenic (colored), luminogenic or fluorogenic compounds which can then be used for detection. This signal can be caused by a label such as a fluorescent or chromogenic label which may be attached to one of the two binding partners, i.e. the truncated Nogo-A polypeptide or the compound of interest, or to a molecule that binds to either of the two binding partners. This signal can also be caused by the change of a physical properties which is caused by the binding, i.e. complex formation itself. An example of such a properties is surface plasmon resonance the value of which is changed during binding of binding partners from which one is immobilized on a surface such as a gold.

Numerous formats for carrying out the method of identifying a compound having detectable affinity exist. A “colony screening” assay (Skerra et al., Anal. Biochem. 196 (1991), 151-155) can, for example, be used if the binding molecule is a polypeptide or peptide. The identification method can also be carried out as a solid phase assay, for example, in an ELISA format, in which the truncated Nogo-A polypeptide of the invention is immobilized in purified form in wells of an ELISA plate and is then brought into contact with the labeled molecule that is suspected to be able to bind to the Nogo-A protein. Such an assay format is more suitable, if binding activity is to be improved based on a compound with known but only weak binding activity. It is however also possible to label the truncated Nogo-A protein for detection of a possible complex formation.

Preferably, the compound having binding affinity to the Nogo-A protein also has a neutralizing effect on the neurite-growth-inhibiting activity of Nogo-A so that the compound may not only be used for diagnostic purposes (where pure binding without neutralizing effect can be sufficient, if tissue staining is desired, for example) but potentially also as pharmaceutical.

In case polypeptides or peptides with detectable binding affinity are to be found by use of the method of the invention, these peptides or polypeptides are preferably subjected to mutagenesis before contacting them with the Nogo-A protein in step a). This mutagenesis can either be a site-directed mutagenesis in which only one or a small number of amino acids are replaced by predetermined amino acids or a partially or entirely random mutagenesis, the latter leading to a library of protein or peptide mutants (muteins) (see Examples). Various strategies for mutagenesis are known to the skilled person in the field of combinatorial biology in order to create such a library.

If nucleic acids such as aptamers are employed as the compound of interest in the identification method of the present invention, they can of course also be employed in form of a library containing a large number of sequence variants. Likewise, also libraries of small organic molecules can be used in the method of identifying molecules having binding affinity to Nogo-A.

Examples of nucleic acids that can be used in a screening for a compound having binding activity to a Nogo-A protein are RNA- or DNA-molecules such as Spiegelmers® described in WO 01/92655, for example. Spiegelmers®) are mirror-image nucleic acids that are supposed to bind to and block a biological target with high affinity and specificity, comparable to an antibody.

If a proteinaceous molecule or a nucleic acid is to be identified as binding compound, the inventive method can comprise the step of enriching at least one mutant nucleic acid or mutein resulting from the mutagenesis and having detectable binding affinity to the Nogo-A protein by screening or selection and/or isolating said at least one mutein or mutant nucleic acid.

Preferred proteinaceous binding molecules that are used in a screening are chosen from the group consisting of antibodies or muteins based on a polypeptide of the lipocalin family. Examples of other proteinaceous binding molecules are the so-called glubodies described in the international patent application WO 96/23879, proteins based on the ankyrin scaffold (Hryniewicz-Jankowska, A. et al., (2002) Folia Histochem. Cytobiol. Vol. 40. 239-249) or crystalline scaffold (WO 01/04144, DE 199 32688) and the proteins described in Skerra (2000) J. Mol. Recognit. 13, 167-187.

An antibody may be used in any of the various forms of known (recombinant) fragments, e.g. as F_{ab} fragment, single-chain F_{V} fragment, F_{V} fragment or diabody, all of which are well known to the person skilled in the art.

In a preferred embodiment of the identification method of the invention, the antibody mutant(s) used is (are) derived from the antibody IN-1 (cf. Examples). However, every antibody which is available in recombinant form or has been raised using the conventional immunization protocol of Köhler and Milstein (Nature 256 (1975), 495497) can be tested for its binding properties. Also libraries, synthetic or from natural sources, which contain a large number of antibody muteins (usually more than approximately 1·10⁷ sequence variants) can be employed for the identification of molecules with detectable affinity to the Nogo-A protein. Such libraries are commercially available, for example, from Cambridge Antibody Technology, Cambridge, UK.

The lipocalin mutein is preferably an anticalin® as described in the German Offenlegungsschrift DE 197 42 706 or the international patent publication WO 99/16873; which is a polypeptide exhibiting specific binding characteristics for a given ligand, like antibodies (cf. also Beste et al., Proc. Natl. Acad. Sci. USA, 96 (1999) 1898-1903).

This lipocalin mutein is based on a member of the lipocalin family in which amino acid positions are mutated in the region of at least one of the four peptide loops, which are arranged at the open end of the cylindrical B-sheet structure. Preferably, these regions correspond (as described in WO 99/16873) to those segments in the linear polypeptide sequence comprising the amino acid positions 28 to 45, 58 to 69, 86 to 99 and 114) to 129 of the bilin-binding protein of Pieris brassicae or homologous positions in other lipocalins. Preferably amino acid positions in two, three or all four of these loops are mutated.

Suitable lipocalins that can be used as scaffold for the generation and identification of anticalins® with binding affinity to the Nogo-A protein are the bilin-binding protein (Bbp), the retinol-binding protein (Rbp), the apolipoprotein D (ApoD), the human neutrophil gelatinase-associated lipocalin (hNGAL), the rat α₂-microglobulin-related protein (A2m) and the mouse 24p3/uterocalin (24p3). The use of human scaffolds such as hNGAL or ApoD is preferred for therapeutic applications.

An example of a binding molecule identified by the method of the invention as described here is the antibody fragment named II.1.8 which is derived from the antibody IN-1. The sequence of the variable domain of the light chain (VL) of the antibody fragment II.1.8 is shown as SEQ ID NO: 12. The sequence of the variable domain of the heavy chain (VH) of II.1.8 is identical to the sequence of IN-1 (Bandtlow et al, 1996, supra) and is shown in SEQ ID NO: 11. The antibody fragment II.1.8 shows improved affinity to the Nogo-A protein, thus allowing detection of Nogo-A in immunochemical experiments, for example.

For its use as diagnostic reagent the binding compound or molecule can be employed in a labeled form. In general, it is possible to label a binding compound such as the antibody fragment II.1.8 with any appropriate chemical substance or enzyme, which directly or indirectly generates in a chemical, enzymatic or physical reaction a detectable compound or a signal that can be used for detection. An example for a physical reaction is the emission of fluorescence after excitation with radiation or the emission of e.g. α- or β-radiation by a radioactive label; alkaline phosphatase, horseradish peroxidase or β-galactosidase are examples of enzyme labels which catalyse the formation of chromogenic (colored), luminogenic or fluorogenic compounds which can then be used for detection. It is noted in this respect, that all of these labels discussed with respect to the (diagnostic) use of a binding compound can, of course, also be employed as signaling means in the method of identifying a binding compound of the invention.

The binding molecule can also be conjugated to a label such as an enzyme label, radioactive label, fluorescent label, chromogenic label, luminescent label, a hapten, biotin, digoxigenin, metal complexes, metals, and colloidal gold. Generally all labels which are used for antibodies, except those which are exclusively used in conjunction with the sugar moiety in the Fc part of immunoglobulins can also be used for conjugation to the muteins of the present invention. These conjugates can be prepared by methods known to the person skilled in the art. Alternatively, a proteinaceous binding compound identified by the method of the present invention can also be produced as chimera, for example, as fusion protein with an enzyme that catalyses a chromogenic or fluorogenic reaction (e.g. alkaline phosphatase, horseradish peroxidase, glutathione-S-transferase). Proteins with inherent chromogenic or fluorescent properties such as the green fluorescent protein (GFP) are suitable fusion partners, too.

The invention is further illustrated by the following examples and the attached drawings in which:

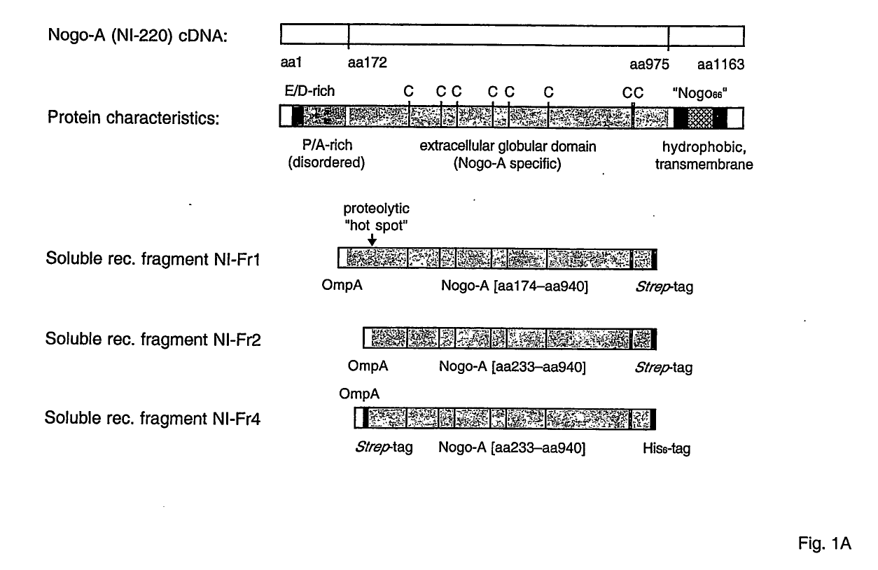

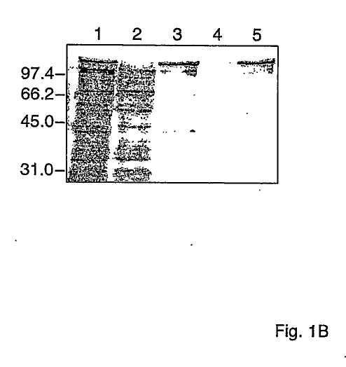

FIG. 1 shows recombinant Nogo-A fragments of the present invention;

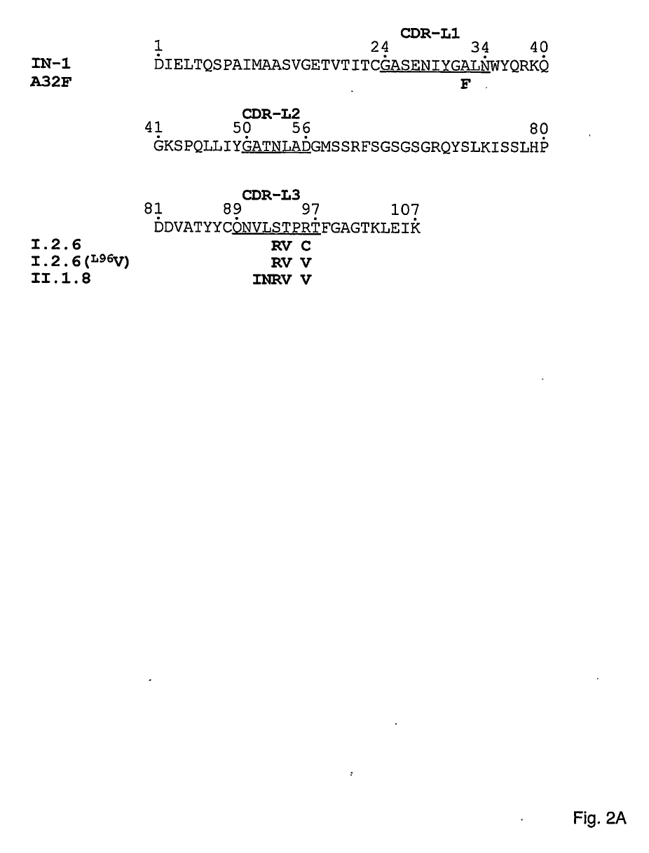

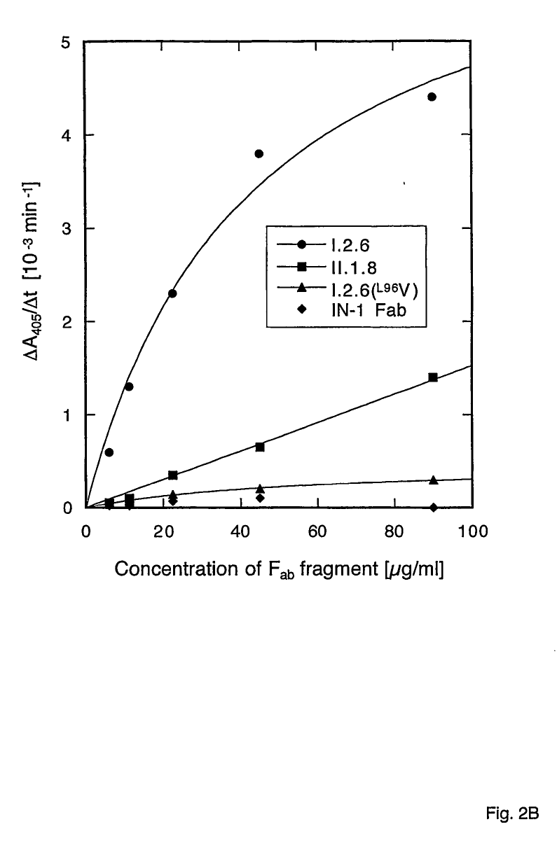

FIG. 2 shows structural and functional characteristics of engineered IN-1 F_{ab} fragments as examples for binding molecules obtained by the method of the invention for identifying a compound having detectable and improved affinity to a Nogo-A protein;

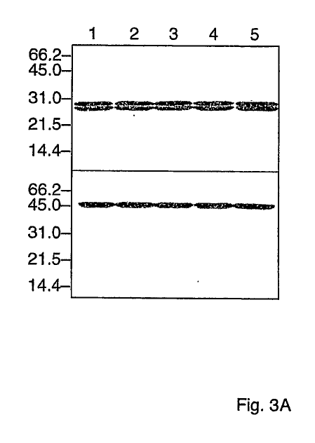

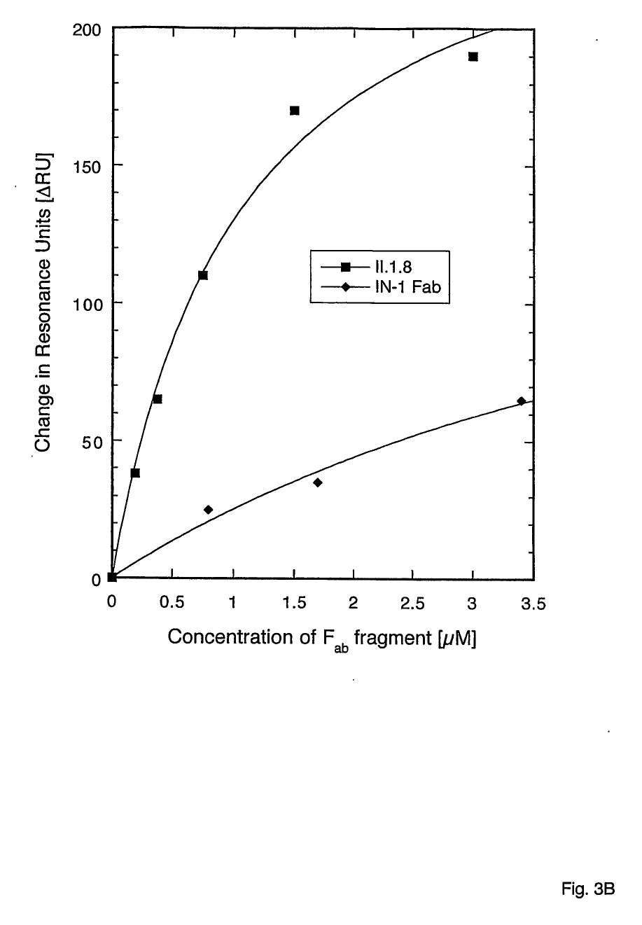

FIG. 3 shows an SDS PAGE of purified IN-1 F_{ab} fragments as well as the antigen affinity determination for the wild-type IN-1 F_{ab} fragment and its mutants by surface plasmon resonance (SPR);

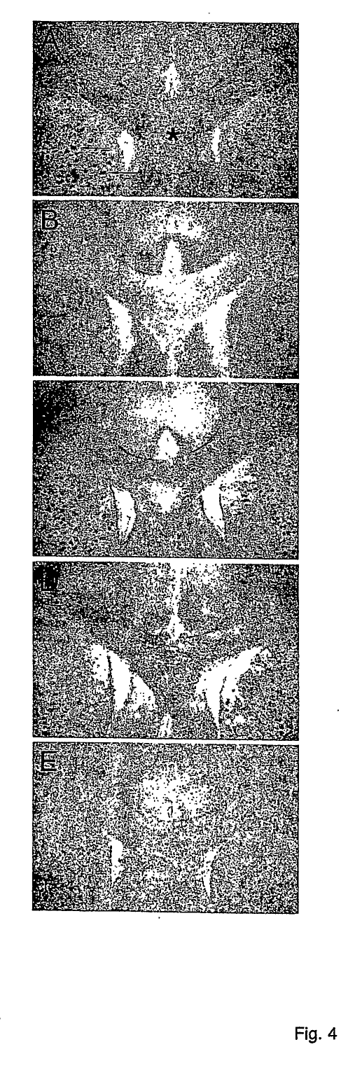

FIG. 4 depicts the specific staining of myelin-rich regions in the rat brain using the IN-1 F_{ab} fragment and its engineered mutants;

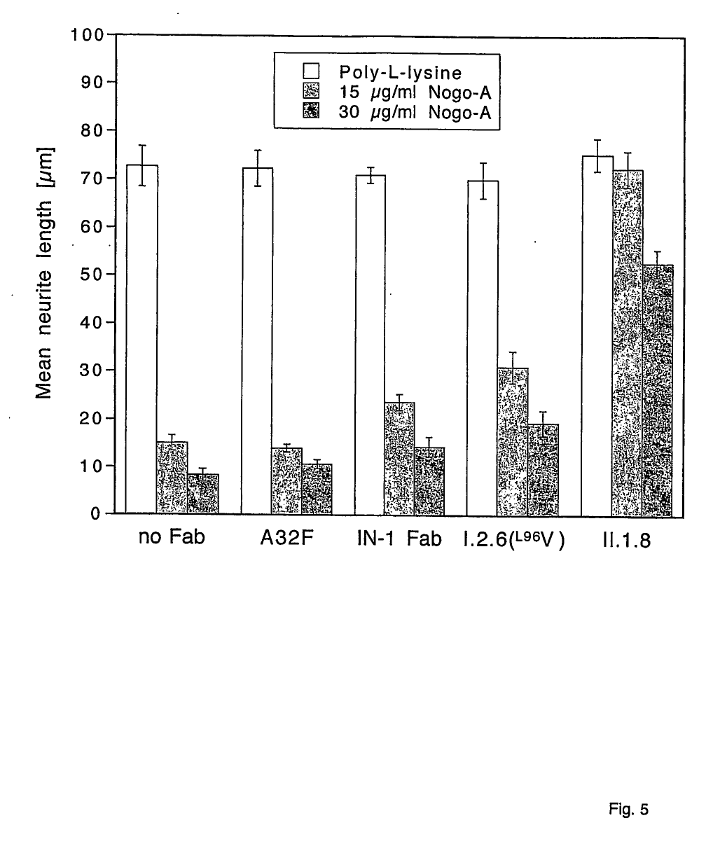

FIG. 5 shows the stepwise improvement of the biological activity of the IN-1 F_{ab} fragment during affinity maturation as determined in an in vitro neurite outgrowth assay;

FIG. 6 shows the amino acid sequences of the full length Nogo-A protein of rat and human origin using the standard one letter code;

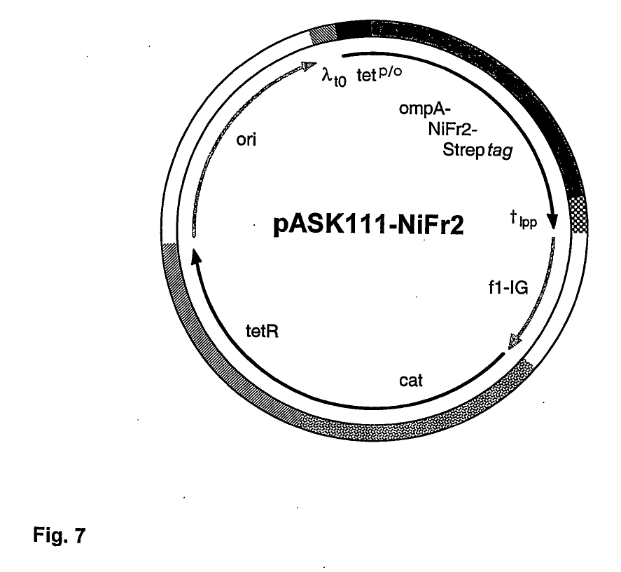

FIG. 7 schematically depicts the expression vector pASK11-FR2.

FIG. 1A schematically shows the structural characteristics of the native neurite growth inhibitor Nogo-A and of examples of recombinant soluble truncated fragments derived from it in the present invention. The fragment NI-Fr1 consists of the amino acids 174 to 940 of the full length Nogo-A rat protein with the Strep-Tag® fused to its C-terminus. The fragment NI-Fr2 consists of the amino acids 223 to 940 of the full length Nogo-A rat protein with the Strep-Tag® fused to its C-terminus. The fragment Ni-Fr4 consists of amino acid 223 to 940 of the full length Nogo-A rat protein equipped with the Strep-Tag® at its N-terminus and a hexa-histidine-tag (His₆) at its C-terminus. FIG. 1B shows a SDS-PAGE analysis of the bacterially produced truncated fragment NI-Fr4. The periplasmic protein extract from E. coli JM83 harbouring pASK111-NIFr4 was loaded in lane 1. The flow-through of an IMAC column is shown in lane 2, eluted protein from IMAC column as applied to the streptavidin column in lane 3, flow-through of streptavidin column in lane 4, purified protein after streptavidin affinity chromatography in lane 5. Molecular sizes are indicated at the left. The proteins were visualized by staining with Coomassie Brilliant Blue.

FIG. 2A shows the amino acid sequence of the V_{L} domain (Kabat database accession no. 029919) of the monoclonal antibody IN-1 together with the substitutions introduced in the course of affinity maturation. Complementarity-determining regions (CDRs) are underlined according to the definition by Kabat et al. Sequences of proteins of immunological interest, 5th Ed. National Institutes of Health, Bethesda Md. (1991), while amino acid positions are numbered consecutively. The mutations obtained by exchange of residues within CDR-L1 and CDR-L3 in the present invention are marked with bold letters below the wild-type sequence. FIG. 2B shows a comparison of the antigen-binding activities of engineered F_{ab} fragments in the ELISA experiments of Examples 5 and 6. Binding of the mutants I.2.6 (circles), II.1.8(squares) and I.2.6(^{L96}V) (triangles) was compared with the binding of the wild-type IN1-F_{ab} fragment (rhombs) to recombinant NI-FR2. The mutants I.2.6 and II.1.8 bind the truncated Nogo-A protein clearly in a concentration-dependent manner, whereas wild-type IN1-F_{ab} fragment does not give rise to a significant binding signal.

FIG. 3A shows an SDS/PAGE analysis of purified recombinant F_{ab} fragments prepared according to the invention. F_{ab} fragments were produced in E. coli JM83 harbouring the corresponding derivative of the vector pASK88 and purified by IMAC. Samples in the upper part were reduced with β-mercaptoethanol prior to SDS gel electrophoresis whereas those in the lower part were kept unreduced: IN-1 (wild-type) F_{ab} fragment is shown in lane 1, the Ala^{L32}ØPhe mutant in lane 2, the I.2.6 mutant in lane 3; the I.2.6(^{L96}V) mutant in lane 4; and the II.1.8 mutant in lane 5. Molecular sizes are indicated at the left. All F_{ab} fragments appear as a homogeneous protein with stoichiometric presence of the light and heavy chains and show quantitative formation of their interchain disulphide bond. FIG. 3B shows the measurement of the concentration-dependent interaction between the IN-1 F_{ab} fragment (rhombs) and its optimized mutant II.1.8 (squares) with the recombinant Nogo-A fragment NI-Fr4 (immobilized on an Ni/NTA-sensor chip® at 285 to 305 ΔRU) by SPR (surface plasmon resonance) technique. Equilibrium values (differences in resonance units, ΔRU) determined after subtraction of the background signal in the absence of NI-Fr4 were plotted against the applied concentration of wild-type IN-1 F_{ab} fragment or its II.1.8 mutant and finally fitted by non-linear regression.

FIG. 4 shows the specific staining of myelin-rich regions in the rat brain. The staining in FIG. 4A was performed with an anti-MOG F_{ab} fragment; the myelinated, MOG-positive Corpus callosum is marked by an asterisk and myelinated fibers of the Capsula interna in the Corpus striatum are indicated by arrows. FIG. 4B shows staining with wild-type IN-1 F_{ab} fragment, FIG. 4C with I.2.6(^{L96}V) F_{ab} fragment, and FIG. 4D with II.1.8 F_{ab} fragment. FIG. 4E shows staining with an anti-CD30 F_{ab} fragment as negative control. Bound F_{ab} fragment was detected in each case with a goat anti-human C_{κ} antibody conjugated with alkaline phosphatase and revealed using the “Fast Red” procedure.

FIG. 5 depicts a graphical representation of the stepwise improvement of the biological activity of the IN-1 F_{ab} fragment during affinity maturation. The columns show the mean neurite lengths of granula cells from the rat cerebellum cultured on a recombinant Nogo-A substrate—or just on poly-L-lysine as a control—whose inhibitory properties were neutralized in the presence of the IN-1 F_{ab} fragment and its engineered mutants (applied at 100 μg/ml). Error bars correspond to standard deviations from triplicate experiments.

FIG. 6A shows the amino acid sequence of the full length Nogo-A protein from rat described by Chen et al, supra. FIG. 6B shows the amino acid sequence of the human full length Nogo-A protein described by GrandPré, et al., supra.

FIG. 7 shows a drawing of pASK111-NiFr2. This vector codes for a fusion protein made of the OmpA-signal sequence and the truncated Nogo-A fragment NI-Fr2 consisting of the amino acids 223 to 940 of the full length Nogo-A rat protein with the Strep-Tag® fused to its C-terminus (cf. FIG. 1a). The entire structural gene is subject to the transcriptional control of the tetracycline promoter/operator (tet^{p/o}) and ends at the lipoprotein transcription terminator (tₗₚₚ). Further elements of the vector are the origin of replication (ori), the intergenic region of the filamentous bacteriophage fl (fl-IG), the chloramphenicol resistance gene (cat) coding for chloramphenicol acetyl transferase and the tetracycline repressor gene (tetR). A relevant segment from the nucleic acid sequence of pASK111-NiFr2 is reproduced together with the encoded amino acid sequence in the sequence protocol as SEQ ID NO: 13. The segment begins with the XbaI-restriction site and ends with the HindIII restriction site. The vector elements—with the exception of the cat gene—outside this region are identical with the vector pASK75, the complete nucleotide sequence of which is given in the German patent publication DE 44 17 598 A1.

### EXAMPLES

### Example 1

### Vector Construction for Nogo Fragments

Unless otherwise indicated, genetic engineering methods known to the person skilled in the art were used, as for example described in Sambrook et al. (supra).

A 2.3 kcbp Nogo-A gene fragment was amplified from the cloned rat cDNA (Chen et al., supra) via PCR with the primers 5′-GCT CAG CGG CCG AGA CCC TTT TTG CTC TTC CTp(S)G-3′ (SEQ ID NO: 3) (the EagI restriction site is underlined) and 5′-GCT TTT AAC TAT GCT GCC CAT TTC TGp(S)T-3′ (SEQ ID NO: 4). The single PCR product was digested with EagI, purified from a 1% agarose gel, and inserted into the multiple cloning region of pASK111 (Vogt and Skerra, J. Mol. Recognit., 14,(2001) 79-86), which had been cut with BsaI (resulting in a sticky end compatible with EagI) as well as Eco47III, yielding pASK111-NiFr1. In this vector the Nogo-A fragment is precisely fused at its N-terminus (i.e. in front of residue 174) to the OmpA signal peptide. This vector leads to the production of a mature protein with a molecular mass of 85.0 kDa, including the Strep-tag at the C-terminus, after processing of the OmpA signal peptide fused in frame to the N-terminus. The vector pASK111-NiFr2 was constructed from pASK111-NiFr1 (SEQ ID NO: 14) by precisely deleting the N-terminal 59 codons from the cloned Nogo-A gene fragment via site-directed mutagenesis using the oligodeoxynucleotide 5′-GGT ATC CAT GTT CTT TAA AAG AGG CCT GCG CTA CGG TAG C-3′ SEQ ID NO: (SEQ ID N NO: 5). Cys residues were replaced by Ser via site-directed mutagenesis with single-stranded DNA prepared from pASK111-NiFr2 using appropriate oligodeoxynucleotide primers.

The C-terminal Strep-tag encoded on pASK111-NiFr2 was exchanged by a His₆ affinity tag by site-directed mutagenesis with the oligodeoxynucleotide 5′-CAC TTC ACA GGT CAA OCT TAT TAA TGG TGA TOG TGA TGG TGA GCG CTT TTA ACT ATG CTG CCC-3′ (SEQ ID NO: 6). A KasI restriction site was concomitantly introduced at the 5′-end of the cloned Nogo-A structural gene using the oligodeoxynucleotide 5′-GGT ATC CAT GTT CTT TAA AAG AGG CGC CCT GCG CTA CGG TAG C-3′ (the KasI recognition site is underlined) (SEQ ID NO: 7), resulting in the vector pASK111-NiFr3. The region encoding the Nogo-A fragment together with the His₆ tag was finally subcloned via KasI and NsiI (cutting within the vector, downstream of the Cam^{r} gene) on pASK-EBA4 (Skerra and Schmidt, (2000) Methods Enzymol., 326A, 271-304), which provided the sequence for an N-terminal Strep-tag II directly downstream of the OmpA signal sequence. The resulting vector was dubbed pASK111-NiFr4 (SEQ ID NO: 15).

Starting from the human cloned cDNA, the analogous procedure was carried out for cloning of the Nogo-A gene fragments. In doing so, the following gene fragments comprised in the vector pASK75strepII (which differ from the vector pASK75 described in DE 44 17 598 A1 only by use of a sequence coding for the StrepTag® II (Schmid et al, supra) instead of the StrepTag ) were obtained: (1.) A fragment encoding the amino acids 246 to 966 of the full length Nogo-A fused at its N-terminus to the OmpA signal peptide with the introduction of an additional aspartate codon in between (i.e. in front of residue 246) and fused at its C-terminus to the Strep-tag II. (2.) A fragment encoding amino acids 334 to 966 of the full length Nogo-A fused at its N-terminus to the OmpA signal peptide with the introduction of an additional glutamine codon in between (i.e. in front of residue 246) and fused at its C-terminus to the Strep-tag II

### Example 2

### Bacterial Production of Soluble Nogo-A Fragments (a Soluble Nogo-A Domain)

By use of the vector pASK111 for the production of Nogo-A fragments of the invention, the respective Nogo-A fragment was fused at its N-terminus to the OmpA signal peptide, thus effecting secretion into the bacterial periplasm, where efficient disulphide bond formation is favoured by an oxidizing redox environment. As explained in Example 1, in case of the rat protein, the bacterial signal peptide was precisely fused to the N-terminus, i.e. residue 174 and 233, respectively, whereas an intermediate amino acid was present between the N-terminal amino acid of the human truncated protein (residue 246 and 334, respectively) and the C-terminus of the signal peptide. At the C-terminus (i.e. following residue 940 of the rat protein, and residue 966 of the human protein) the fragment was fused with the Strep-tag affinity peptide, conferring binding activity towards streptavidin for simplified purification. Transcription of the resulting hybrid gene was under tight control of the tetracycline promoter/operator.

Cultures of E. coli JM83 transformed with the respective expression vector pASK111 obtained in Example 1 were grown in 2 l Luria-Bertani (UB) medium supplemented with chloramphenicol as antibiotic at 22° C. and 200 rpm. Gene expression was induced at an optical density of 0.5 at 550 nm by addition of 400 μg/L anhydrotetracycline (aTc; Acros Organics, Geel, Belgium). After 3 h induction the bacteria were harvested by centrifugation and the periplasmic protein fraction was prepared as described by Skerra and Schmidt, supra, with the exception that 200 μg/ml lysozyme were also added to the cell fractionation buffer (50 mM NaPᵢ, pH 7.5, 500 mM sucrose, 1 mM EDTA) for improved release of the Nogo-A fragments.

All Nogo-A fragments (NI-Fr1 (SEQ ID NO: 16), NI-Fr2 (SEQ ID NO: 17)) of the rat protein as well as corresponding human polypeptides) were purified from the periplasmic protein extract via the Strep-tag fused to their C-termini employing streptavidin affinity chromatography (Skerra and Schmidt, supra), whereby elution was effected under mild conditions in the presence of desthiobiotin. After dialysis against chromatography buffer (50 mM NaPᵢ, pH 7.5, 150 mM NaCl, 1 mM EDTA) and concentration (Vivaspin 15, MWCO 30 kDa; Greiner, Frickenhausen, Germany) of the eluate further purification was achieved by gel filtration on a Superdex 200 prep grade column (Pharmacia, Uppsala, Sweden) using Dynamax SD-300 HPLC equipment (Rainin, Woburn, Mass.). NI-Fr4 (SEQ ID NO: 18) was first purified by means of the His₆ tag via IMAC (Skerra, Gene, 141, (1994a) 79-84) using 50 mM NaPᵢ, pH 7.5, 1 M NaCl as chromatography buffer and a linear elution gradient from 0 to 75 mM imidazole.HCl. The specifically eluted protein fraction was then subjected to streptavidin affinity chromatography as above.

The yields of purified recombinant rat Nogo-proteins from 2 L shaker-flask experiments were highly reproducible and varied between 0.1 and 0.3 mg L⁻¹ OD⁻¹ for the Nogo-A fragments. After purification the proteins were stored in PBS (4 mM KH₂PO₄, 16 mM Na₂HPO₄, 115 mM NaCl) containing 0.1 mM EDTA at 4° C. for up to several weeks. Protein purity was checked by SDS-PAGE using 0.1% (w/v) SDS, 10% or 15% (w/v) polyacrylamide gels (ing and Gregerson, (1986) Anal. Biochem., 155, 83-88) stained with Coomassie brilliant blue. The concentration of the purified recombinant proteins of rat origin was determined using calculated absorption coefficients at 280 nm (Gill and von Hippel, (1989) Anal. Biochem., 182, 319-326) of 0.41 ml mg⁻¹ cm⁻¹ for the Nogo-A fragments.

As shown in FIG. 1, the polypeptide comprising residues 174 to 940 (containing 767 residues, i.e. 66% of full length Nogo-A) was first used for production as a recombinant protein.

Upon induction of gene expression NI-Fr1. (SEQ ID NO: 16) was readily liberated from the periplasmic protein fraction of E. coli and purified by streptavidin affinity chromatography in one step. SDS PAGE analysis revealed that ca. 50% of the recombinant protein comprised a product with the proper length whereas 50% corresponded to a series of smaller polypeptides, probably representing proteolytic degradation products (not shown). In particular, there appeared one prominent band just underneath that for the major recombinant protein. Both bands were subjected to N-terminal sequencing. The upper band yielded the sequence Glu-Thr-Leu-Phe-Ala, which resulted from the precise cleavage of the OmpA signal peptide. The lower band started with the amino acids Ser-Phe-Lys-Glu-His, i.e. at a position 59 codons downstream within the cloned sequence (beginning at residue 233 in the full length primary structure). Its appearance was most likely due to the action of a bacterial protease and might indicate that the N-terminal part of the chosen Nogo-A fragment still belongs to a polypeptide segment devoid of well-defined structure.

In order to achieve better homogeneity of the gene product the first 59 residues of the mature polypeptide chain were deleted from the cloned coding region, leading to NI-Fr2 (SEQ ID NO: 17) (cf. FIG. 1A). This protein was readily produced in the periplasm of E. coli, with similar yields as the former version but clearly reduced degradation pattern. The possible presence of structural disulphide bonds in the recombinant protein was investigated by individually substituting all eight Cys residues (corresponding to positions 323, 403, 443, 536, 574, 676, 885, and 890 in the full length Nogo-A sequence) with Ser via site-directed mutagenesis. The eight mutant Nogo-A fragments were produced in E. coli as before. However, it was not possible to recover the mutants Cys³²³ØSer and Cys⁸⁸⁵ØSer from the periplasmic protein fraction, while the mutants Cys⁴⁴³ØSer and Cys⁸⁹⁰ØSer gave rise to significantly diminished yields after Strep-tag purification when compared with the wild-type protein. In contrast, the other four mutants were produced at similar amounts as the original Nogo-A fragment. These observations indicate that at least some of the Cys residues are important for folding and may be involved in cystine crosslinks.

The wild-type NI-Fr2 (SEQ ID NO: 17) protein still gave rise to certain truncated products, which was considered undesirable for precise binding measurements (see below). Therefore, a doubly tagged version of the recombinant protein was prepared using an otherwise identical expression system. First, the Strep-tag at the C-terminus was exchanged by a His₆-tag (yielding NI-Fr3 as an intermediate construct, not shown), and, second, the Strep-tag was inserted at the N-terminus again, downstream of the OmpA signal peptide. Interestingly, the yield of bacterially produced soluble protein, termed NI-Fr4 (SEQ ID NO: 18) (cf. FIG. 1A), was found to be significantly higher (by a factor of 2.5, approaching 300 μg L⁻¹ OD⁻¹). NI-Fr4 (SEQ ID NO: 18) was isolated from the periplasmic protein fraction in two steps by immobilized metal affinity chromatography (IMAC) followed by streptavidin affinity chromatography as described above. This protein was essentially pure, just a minor fraction of truncated polypeptide chains was still detectable (FIG. 1B).

Furthermore, a mutant of NI-Fr2 devoid of Cys⁵⁷⁴ and Cys⁶⁷⁶ was also produced as described above and used as Nogo protein in the affinity maturation of antibody fragments directed to Nogo-A (Example 5).

Thus, the invention provides for the first time soluble and stable Nogo-A polypeptides which can be used for the detailed elucidation of the biological role of the Nogo-A protein and in the identification of substances with binding affinity to Nogo-A. This identification method will be demonstrated in the following Examples.

### Example 3

### Identification of Antibody Fragments Derived from IN-1 with Improved Binding Affinity to Nogo-A

The IN-1 F_{ab} fragment with variable domains derived from the mouse monoclonal antibody IN-1 (Bandtlow et al., 1996, supra) and human constant domains belonging to the subclass IgG1/κ (Schiweck and Skerra, (1995) Proteins: Struct. Funct. Genet., 23, 561-565) was used as starting molecule for the identification of antibody fragments with improved affinity and neutralizing effect on the neurite-growth-inhibiting activity of Nogo-A. The IN-1 muteins used in the method of identifying new binding molecules were either derived from a computer-based modeling study or an evolutionary approach. The following general methodology was used for construction of the respective genes and the production antibody fragments.

Vector Construction for F_{ab} Fragments

The IN-1 F_{ab} fragment and its mutants were produced utilizing the vectors pASK88, pASK106 or pASK107. All of them encode a chimeric F_{ab} fragment with variable domains derived from the mouse monoclonal antibody IN-1 and human constant domains belonging to the subclass IgG1/κ (see above). Secretion into the oxidizing milieu of the bacterial periplasm is ensured by the presence of signal peptides at the N-termini of both chains (Skerra, 1994a, supra) and transcription of the artificial dicistronic operon is under tight control of the chemically inducible tet^{p/o} (Skerra, Gene, (1994b) 151, 131-135). pASK88 (Schiweck and Skerra, supra) was used for soluble expression and purification via the His₆ tag attached to the C-terminus of the heavy chain (Fiedler and Skerra, (2001a) In Kontermann, R. and Dübel, S. (eds.), Antibody Engineering. Springer Verlag, Heidelberg, pp. 243-256; Skerra, 1994b), whereas pASK107 provided the Strep-tag II for streptavidin affinity purification instead. pASK106 codes for a F_{ab} fragment similarly as pASK88 but with an albumin-binding domain (ABD) appended to the C-terminus of the light chain (König and Skerra, (1998) J. Immunol Methods, 218, 73-83). The variable domain genes were exchanged between the differing vector formats using conserved restriction sites as described (Skerra, 1994a).

Single amino acid exchanges within the IN-1 F_{ab} fragment or its mutants were introduced by site-directed mutagenesis. For this purpose single-stranded DNA of the corresponding vectors pASK88-IN1 or pASK88-I.2.6 (see below) was used in conjunction with appropriate oligodeoxynucleotide primers.

Random amino acid substitutions used for the generation of the genetic random library of Example 3.2 were introduced into the variable domain (V_{L}) gene of the IN-1 light chain at defined positions via PCR by means of degenerate oligodeoxynucleotide primers (without the phosphorothioate modification) in conjunction with Taq DNA polymerase. Amplification was performed on pASK85-IN1 with the originally cloned genes (Bandtlow et al., 1996, supra) as template. The forward primer 5′-GAC ATT GAG CTC ACC CAG TCT CCA GCA ATC ATG KCT GC-3′ (SEQ ID NO. 8) (SstI restriction site underlined) was used in all experiments whereas the oligodeoxynucleotide 5′-GCG CTT CAG CTC GAG CTT GGT CCC AGC TCC GAA CGT MNN AGG MNN MNN TAA CACATT TTG ACA GTA-3′ (SEQ ID. NO. 9) (XhoI restriction site underlined) served as backward primer for randomizing the CDR-L3 positions L93, L94, and L96 at the first stage of the affinity maturation process (see below, Example 3.2). The second mutagenesis cycle was performed with pASK88-I.2.6(^{L96}V) as template and the oligodeoxynucleotide 5′-GCG CTT CAG CTC GAG CTT GGT CCC AGC TCC GAA CGT AAC CGG CAC CCG MNN MNN ATT TTG ACA GTA ATA CGT TGC-3′ (SEQ ID NO: 10) as second primer for randomizing the positions L91 and L92 together with fixed mutations at L93, L94, and L96. In each case a single PCR product was obtained, purified from a 1% agarose gel, and cut with SstI and XhoI. The resulting DNA fragment of approximately 300 bp was ligated with the likewise cut vector backbone of pASK106-IN1 (cf. above). Colonies obtained after transformation of CaCl₂-competent E. coli K-12 JM83 cells (Yanisch-Perron et al., (1985) Gene, 33, 103-119) were directly subjected to the filter-sandwich colony screening assay.

Bacterial Production of F_{ab} Fragments

Cultures of E. coli JM83 transformed with the respective derivatives of vectors pASK88, pASK106, and pASK 107 were grown in 2 l Luria-Bertani (LB) medium supplemented with ampicillin at 22° C. and 200 rpm. Gene expression was induced at an optical density of 0.5 at 550 nm by addition of 200 μg/L anhydrotetracycline (aTc; Acros Organics, Geel, Belgium). After 3 h induction the bacteria were harvested by centrifugation and the periplasmic protein fraction was prepared as described by Skerra and Schmidt, supra.

The recombinant IN-1 F_{ab} fragments were purified either by IMAC via the His₆ tag fused to the C-terminus of their heavy chain (Fiedler and Skerra, 2001a, supra) or, when using pASK107 (cf. above), via streptavidin affinity chromatography (Schlapschy and Skerra, (2001) In Kontermann, R. and Dübel, S. (eds.) Antibody Engineering. Springer Verlag, Heidelberg, pp. 292-306). IMAC was also performed under FPLC conditions using a POROS MC/M column (0.46 cm×10 cm; PerSeptive Biosystems, Wiesbaden, Germany) charged with Zn²⁺ ions and Dynamax SD-300 HPLC equipment (Rainin, Woburn, Mass.) operating at a flow rate of 2.0 ml/min. 12.5 ml of periplasmic extract from a 2 L E. coli culture dialyzed against 50 mM NaPᵢ, pH 7.5, 500 mM betaine was applied to the column and, after washing with dialysis buffer, elution was effected by application of a linear gradient of 200 mM imidazole.HCl, pH 7.5, 50 mM NaPᵢ, 500 mM betaine against dialysis buffer. This method enabled a five-fold quicker purification compared with the conventional procedure of Fiedler and Skerra, 2001a, supra, yielding recombinant F_{ab} fragments with an apparent purity of >95% as estimated from SDS-PAGE. The yields of purified recombinant proteins from 2 L shaker-flask experiments were highly reproducible and varied between 0.04 and 0.8 mg L⁻¹ OD⁻¹ for the different F_{ab} fragments.

### Example 3.1

### Identification of Antibody Fragments with Improved Binding Affinity to Nogo-A Based on Computer Modeling

Experiments on the detection of natural Nogo-A on Western blots or on tissue sections by means of the bacterially produced IN-1 F_{ab} fragment revealed relatively weak signals (Bandtlow et al., 1996, supra), indicating that the antigen affinity was poor.

A computer-modeling study was first carried out order to select candidate molecules to be tested in the identification method of the present invention. This modeling study was based on a human anti-thyroid peroxidase autoantibody (Protein Data Bank (PDB) entry 1VGE) and a murine anti-phenylarsonate antibody (PDB entry 6FAB), both of which have sets of CDRs with the same lengths and canonical structure determinants as IN-1 and share a high amino acid sequence similarity with it. This analysis revealed that the CDR-L3 of IN-1 and, to a lesser extent, its CDR-L1 appeared to be the most promising target regions for protein engineering towards improved antigen recognition. Especially residue L96 and also residue L32 (in CDR-L1) appeared to be exposed close to the center of the combining site and thus to be possibly involved in contacts with the antigen.

Within CDR-L1 both IN-1 and 1VGE have an Ala residue at position L32 whereas 6FAB carries a Phe. On the other hand, IN-1 as well as 6FAB carry an Arg at position L96 (in CDR-L3) while 1VGE exhibits a Leu. Therefore, the structural consequences of the amino acid exchanges A^{L32}ØF and R^{L96}ØL within the V_{L} domain of IN-1 were modeled, resulting in their identification as potential paratope residues. The corresponding single amino acid exchanges in the recombinant F_{ab} fragment were introduced by site-directed mutagenesis followed by production in E. coli and purification via IMAC as described above. A test for neutralizing biological activity in the 3T3 fibroblast assay for inhibition of cell spreading on a CNS myelin substrate (Bandtlow et al., 1996, supra) revealed that the mutant R^{L96}ØL had a slightly improved activity. In contrast, the mutant A^{L32}ØF had mostly lost its neutralizing activity when compared with the wild-type IN-1 F_{ab} fragment (data not shown).

Example 3.2

### Identification of Antibody Fragments with Improved Binding Affinity to Nogo-A by in vitro Affinity Maturation of the IN-1 F_{ab} Fragment

In order to perform functionally more complex changes within the paratope of the IN-1 antibody a cluster of three amino acids in CDR-L3 corresponding to positions L93, L94, and L96 was subjected to targeted random mutagenesis.

All 20 side chains were allowed for substitution in each position, followed by screening for improved binding of the recombinant Nogo-A fragment via a filter-sandwich colony screening assay. This assay was carried out based on published procedures (Skerra et al., Anal. Biochem., 196, 151-155; Schlehuber et al., (2000) J. Mol. Biol., 297, 1105-1120).

For this purpose a genetic random library was prepared by PCR amplification of the IN-1 V_{L} gene using the degenerate primer of SEQ ID. NO. 9 that carried the corresponding mixed base positions (see above). The mutagenized gene fragment was recloned on the expression vector pASK106-IN1 (encoding a F_{ab} fragment fused with an albumin-binding domain to the C-terminus of its light chain; König and Skerra, supra). E. coli JM83 was transformed with the ligation mixture and transformed cells harboring the pASK106 vector were plated on a hydrophilic membrane (GVWP, 0.22 μm; Millipore, Bedford, Mass.), placed on a petri dish with LB/Amp agar, such that approximately 500 colonies were obtained, and incubated at 37° C. for 8 to 9 h. In the meantime a hydrophobic membrane (Immobilon-P, 0.45 μm; Millipore) was coated with 10 mg/ml human serum albumin (HSA; Sigma, Deisenhofen, Germany) in PBS for four hours and blocked with 3% (w/v) BSA (Roth, Karlsruhe, Germany), 0.5% (v/v) Tween 20 in PBS. The membrane was washed twice with PBS, soaked in LB/Amp containing 200 μg/ml aTc, and placed on an LB/Amp agar plate supplemented with 200 μg/ml aTc. The first membrane, carrying tiny colonies of the transformed cells, was then placed onto the second (hydrophobic) membrane. The filter sandwich was incubated for 16 h at 22° C. During this period the mutated IN-1 F_{ab} fragments became secreted—and partially released from the colonies by leakage from the bacterial periplasm—and finally immobilized on the lower membrane via complex formation between HSA and ABD.

The first membrane with the still viable colonies was transferred to a fresh LB/Amp agar plate and stored at 4° C. The second membrane was washed three times in PBS containing 0.1% (v/v) Tween 20 (PBS/T) and the immobilized F_{ab} fragments, each in a spot 30 corresponding to the position of the original colony, were probed for antigen binding. To this end recombinant Nogo-A fragment NI-FR2 was labeled at a molar ratio of 5:1 with digoxigenin-3-O-methylcarbonyl-e-aminocaproic acid N-hydroxy-succinimide ester (Roche Diagnostics, Mannheim, Germany) and applied to the membrane for one hour at a concentration of 30 or 50 μg/ml in PBS/T. After washing three times with PBS/T the membrane was incubated for one hour with 0.75 u/ml anti-digoxigenin F_{ab} fragment conjugated with alkaline phosphatase (Roche Diagnostics) in 10 ml PBS/T. The membrane was finally washed twice with PBS/T and twice with PBS and the signals were developed using standard chromogenic substrates as described (Schlehuber et al., supra). Colonies corresponding to signals with an intensity above average were identified, recovered from the first membrane, and propagated for further analysis of their recombinant gene product.

In total, the cell suspension containing transformed E. coli JM83 cells harboring the pASK106 vector was plated on four filter membranes, placed on top of agar plates, thus screening approximately 2000 colonies in parallel. From colonies that gave rise to staining signals above average 31 clones were recovered, propagated, and their plasmids were isolated for DNA sequence analysis. Out of these 31 investigated clones, 12 plasmids were identified carrying functional V_{L} genes (for the mutations see Table 1), whereas otherwise frameshift mutations or internal amber termination codons were abundant.

TABLE I
Mutants obtained from a first affinity maturation based on the IN-1 F_{ab} fragment

| | Position | | | | | Signal in | Expression | ELISA |
|---|---|---|---|---|---|---|---|---|
| | L91 | L92 | L93 | L94 | L96 | CSA^{a} | yield^{b} | signal |
| IN-1 wt | Val | Leu | Ser | Thr | Arg | + | +++ | − |
| I.1.4 | —^{c} | — | Pro | Val | Trp | +++ | + | + |
| I.1.6 | — | — | Asn | Leu | Cys | ++ | | |
| I.1.11 | — | — | Tyr | Thr | Cys | ++ | | |
| I.1.16 | — | — | Met | Cys | Asn | ++ | + | − |
| I.2.2 | — | — | Arg | Thr | Asn | +++ | +++ | − |
| I.2.4 | — | — | Gly | Thr | Phe | +++ | | |
| I.2.5 | — | — | Pro | Cys | Val | +++ | | |
| I.2.6 | — | — | Arg | Val | Cys | +++ | + | +++ |
| I.2.8 | — | — | Tyr | Ala | Gly | ++ | + | − |
| I.2.9 | — | — | Arg | Pro | Pro | ++ | ++ | − |
| I.3.7 | — | — | Phe | Arg | Leu | +++ | + | − |
| I.4.4 | — | — | Asp | Arg | Leu | +++ | | |
| I.2.6 (^{L96}V) | — | — | Arg | Val | Val | | +++ | + |

^{a}filter-sandwich colony screening assay;
^{b}in E. coli JM83 using the vector pASK88;
^{c}no exchange

### Example 4

### Production of IN-1 Muteins

The muteins derived from the variable domains of the antibody IN-1 identified in Example 3.2 were then produced in amounts suitable for characterization of the binding properties of these muteins.

For soluble production of the recombinant F_{ab} fragments in a standard format (i.e. without the ABD domain but still having a His₆ tag fused to the C-terminus of the heavy chain) the mutagenized V_{L} gene cassettes from seven selected clones (cf. Table I) were subcloned on pASK88-IN1 (Fiedler and Skerra, (1999) Protein Expr. Purif., 17, 421-427). The mutants were produced in shaker flask cultures and isolated from the periplasmic protein fraction in one step via IMAC. All F_{ab} fragments contained the light and heavy chains in stoichiometric composition and quantitatively linked via a disulphide bond.

Antigen-binding activity of the mutant F_{ab} fragments was subsequently tested by ELISA using the recombinant NI-Fr2 for coating of the microtitre plate wells (FIG. 2).

ELISA was carried out in a 96 well microtitre plate (Becton Dickinson, Heidelberg, Germany) at ambient temperature with incubation steps of 1 h unless otherwise stated. Three washing steps with PBS/T were used after each incubation, and residual liquid was removed thoroughly. The wells were coated for 4 h with 50 μl of a solution of MI-Fr2 at concentrations between 180 and 200 μg/ml in PBS buffer and then blocked with 200 μl 3% (w/v) BSA, 0.5% (v/v) Tween 20 in PBS. After washing, 50 μl of the purified recombinant F_{ab} fragment was applied at a dilution series in PBS/T. The wells were then incubated with 50 μl anti-human C_{κ} antibody conjugated with alkaline phosphatase (Sigma), diluted 1:1000 in PBS/T. Signals were finally developed in the presence of p-nitrophenyl phosphate (Voss and Skerra, (1997) Protein Eng., 10, 975-982). Enzymatic activity was measured at 25° C. as the change in absorbance at 405 nm per min with a SpectraMAX 250 instrument (Molecular Devices, Sunnyvale, Calif.). The data were corrected for background values determined in wells that were merely coated with BSA and fitted by non-linear least squares regression as described by Voss and Skerra, supra

Almost no binding signal above background was obtained with the recombinant wild-type IN-1 F_{ab} fragment, illustrating its low antigen affinity. In contrast, the mutant I.2.6 (cf. FIG. 2A) gave rise to a clearly detectable and concentration-dependent binding signal. No significant signal was obtained in a control experiment with BSA serving as antigen. Hence, the mutant I.2.6 was the protein of choice for further affinity maturation experiments.

### Example 5

### Affinity Maturation of the Mutant I.2.6

Unfortunately, the I.2.6 mutant of the IN-1 F_{ab} fragment was produced as a soluble protein in E. coli at a much lower level, with a relative yield of 5% after purification (0.04 mg L⁻¹ OD⁻¹ vs. 0.8 mg L⁻¹ OD⁻¹ for the wild-type IN-1 F_{ab} fragment). Obviously, the free Cys residue that occurred at the exposed position L96 within CDR-L3 had a deleterious influence on the folding efficiency of the Ig fragment and a concomitant toxic effect on the bacterial host cell, as it had been similarly observed in other cases. Following earlier substitution experiments concerning position L96 (cf. above) attempts were made to replace the Cys residue in the I.2.6 mutant by small apolar side chains such as those of Ala, Val, Met, Leu, and Ile. The substitutions were introduced by site-directed mutagenesis and all corresponding recombinant F_{ab} fragments were produced and purified as before, resulting in yields that were similar again to the wild type IN-1 F_{ab} fragment. However, when binding activity towards the recombinant NI-Fr2 antigen was tested in an ELISA as described above, all these mutants gave rise to significantly lower signals than the original I.2.6 F_{ab} fragment. Merely the replacement Cys^{L96}ØVal (cf. FIG. 2) resulted in a detectable binding behavior and was therefore used as basis for the second affinity maturation cycle.

CDR-L3 forms a connecting loop between two neighboring beta-strands such that the positions L91 and L92 are in close spatial proximity with L96. Hence, in order to structurally compensate a possible misfit at position L96—due to the exchange of Cys by Val—the positions L91 and L92 within CDR-L3 of the I.2.6(^{L96}Val) F_{ab} fragment were subjected to targeted random mutagenesis using the oligonucleotide of SEQ ID NO: 9 and the filter-sandwich colony screening assay was performed again. This time the stringency of selection was raised by lowering the concentration of the recombinant antigen - a mutant of NI-Fr2 devoid of Cys⁵⁷⁴ and Cys⁶⁷⁶—from 50 μg/ml to 30 μg/ml. From screening approximately 1000 colonies spread on two filter membranes, 16 clones were identified according to their pronounced color signals. In contrast with the previous experiment all of them carried plasmids encoding functional mutants of the I.2.6(^{L96}V) F_{ab} fragment. The V_{L} gene cassettes of four clones (cf. Table 2) were subcloned on pASK88-IN1 and the corresponding F_{ab} fragments were produced and purified as before. One of them, the II.1.8 F_{ab} fragment (cf. FIG. 2A), exhibited clearly improved binding activity over the I.2.6(^{L96}V) mutant in an ELISA (FIG. 2B), even though its affinity was still lower than that of the original I.2.6 mutant carrying the free Cys residue. Nevertheless, the yield of the II.1.8 mutant was 12-fold higher upon expression in E. coli and thus close to that of the recombinant wild-type IN-1 F_{ab} fragment (0.5 vs. 0.8 mg L⁻¹ OD⁻¹, respectively).

TABLE 2
Mutants obtained from a second affinity maturation
based on the I.2.6-F_{ab} fragment

| | Position | | | | | Signal in | Expression | ELISA |
|---|---|---|---|---|---|---|---|---|
| | L91 | L92 | L93 | L94 | L96 | CSA^{a} | yield^{b} | signal |
| IN-1 wt | Val | Leu | Ser | Thr | Arg | + | +++ | − |
| I.2.6 (^{L96}V) | — | — | Arg | Val | Val | | +++ | + |
| II.1.1 | Arg | Lys | Arg | Val | Val | +++ | +++ | − |
| II.1.3 | Met | Lys | Arg | Val | Val | ++ | +++ | − |
| II.1.7 | Leu | Lys | Arg | Val | Val | ++ | +++ | − |
| II.1.8 | Ile | Asn | Arg | Val | Val | ++ | +++ | ++ |

^{a}filter-sandwich colony screening assay;
^{b}in E. coli JM83 using the vector pASK88;
^{c}no exchange

### Example 6

### Functional Analysis of Engineered F_{ab} Fragments

For a detailed analysis of the antigen-binding activity and application in immunohistochemistry as well as cell culture assays the different engineered versions of the IN-I F_{ab} fragment were produced in E. coli in shaker flask cultures and purified by IMAC to homogeneity (FIG. 3a).

The thermodynamic affinity for the recombinant Nogo-A fragment NI-Fr4 was determined both for the II.1.8 mutant and for the wild-type IN-1 F_{ab} fragment using the method of real time surface plasmon resonance (SPR) on a Biacore-X® system equipped with an Ni/NTA-derivatized sensor chip® (Biacore AB, Uppsala, Sweden). PBS containing 0.005% (v/v) surfactant P20 was used as continuous flow buffer as well as for dilution of proteins. Analysis was performed at 25° C. using a flow rate of 35 μl/min.

For each measurement the derivatized chip surface was charged with 70 μl 0.5 mM NiSO₄, followed by immobilization of NI-Fr4 via its His₆ tag in one of the two flow channels by applying 70 μl of a 50 μg/ml solution of the purified recombinant protein.

Then the F_{ab} fragment (produced by means of the vector pASK107 and purified via the Strep-tag II; see Example 1)) was injected at a defined concentration (between 0.25 and 6.8 μM) for 2 minutes, followed by buffer flow for 4 minutes. The chip surface was regenerated using 70 μl 0.35 M EDTA, pH 8.0 in flow buffer prior to the next measurement. Each time-dependent binding isotherm of the F_{ab} fragment was corrected for the background signal that was detected in the flow channel without NI-Fr4 using BIAevaluation software (Version 3.0). Resonance unit values for the bound F_{ab} fragment at equilibrium for each applied concentration were then deduced and fitted (Voss and Skerra, supra) by non-linear least squares regression using an equation of the type y=a*x/(b+x).

By this way binding isotherms were obtained for the wild-type and engineered F_{ab} fragments (FIG. 3B), from which dissociation constants were deduced. The K_{D} value for the recombinant wild-type IN-1 F_{ab} fragment was 7.8±1.9 μM. In contrast, the dissociation constant for its II.1.8 mutant was 1.04±0.18 μM, i.e. 8-fold better. Control experiments with an unrelated protein, recombinant cystatin carrying a His₆-tag, that was used instead of the Nogo-A fragment for coating of the sensor chip confirmed absence of unspecific binding (not shown).

### Example 7

### Use of Engineered IN 1-F_{ab} Fragments for Detection of Natural Nogo-A

The engineered II.1.8 F_{ab} fragment was further employed for the detection of natural Nogo-A by immunohistochemistry.

For this purpose, cryosections (12 μm) of rat brain (Rattus norvegicus) were fixed for 10 minutes using ice-cold ethanol. The following incubation steps were then each performed for 1 h at room temperature in a humid chamber using PBS. Unless otherwise stated slides were washed for 5 min with PBS. After blocking with 4% (w/v) BSA the F_{ab} fragment (produced using the pASK88 vector type and purified via the His₆ tag) was applied at a concentration of 100 μg/ml. After three washing steps bound F_{ab} fragment was detected with an anti-human Cₖ antibody alkaline phosphatase conjugate (Sigma), diluted 1:100. The sections were then washed three times with TBS (25 mM Tris/HCl, pH 7.4, 145 mM NaCl, 3 mM KCl) and staining was performed using a “Fast Red” kit (Roche Diagnostics). The microscopic slides were photographed on an Axiophot microscope (Carl Zeiss, Jena, Germany) using 10- or 20-fold magnification.

FIG. 4 shows cross sections of adult rat brain which were stained with different recombinant F_{ab} fragments, followed by the above-mentioned secondary antibody conjugated with a reporter enzyme. The II.1.8 mutant specifically stained the myelinated regions, especially the Corpus callosum and transected fiber bundles of the Capsula interna in the Corpus striatum. The staining pattern is similar in morphology and intensity to the one obtained with a recombinant F_{ab} fragment derived from the monoclonal antibody 8-18C5, which is directed against the major oligodendrocyte glycoprotein MOG (Linington et al., (1984) J. Neuroimmunol., 6, 387-396). The staining with the recombinant wild-type IN-1 F_{ab} fragment was very weak under the present conditions of fixation. An unrelated recombinant anti-CD30 F_{ab} fragment derived from the HRS-3 antibody (Engert et al., (1990) Cancer Res., 50, 2929-2935) gave only background staining. These results demonstrate that the affinity of the II.1.8 mutant of the IN-1 F_{ab} fragment has been raised by use of truncated Nogo-proteins of the invention to a sufficient extent in order to detect the Nogo-A antigen in standard immunochemical experiments. Analogous data were obtained using immunofluorescence microscopy (not shown).

### Example 8

### Neutralization of the Neurite-Growth-Inhibiting Activity of Nogo-A by Engineered IN 1-F_{ab} Fragments

Finally, the engineered F_{ab} fragments were tested for their neutralizing effect on Nogo-A substrate properties using a cell culture assay.

Neurite growth-modulating properties of the different F_{ab} fragments were tested on 4-well plastic dishes (Greiner, Nürtingen, Germany) coated with recombinant Nogo-A. The test wells were coated for 20 min with 100 μg/ml poly-L-lysine, washed with Hank's balanced salt solution (HBSS; Life Technologies, Basel, Switzerland) and coated for 2 h with 15 or 30 μg/ml of recombinant rat Nogo-A (Chen et al., supra) Recombinant Nogo-A was omitted in the wells serving for control. After aspiration, the wells were washed with Dulbecco's modified Eagle's medium (DMEM; Life Technologies) containing 10% v/v fetal calf serum (FCS; Life Technologies) and blocked in the same medium for 20 min at 37° C.

Cerebellar cell cultures were prepared from rat cerebella on postnatal day 7/8. Cells were dissociated by combined trituration and trypsinization and purified on Percoll gradients as described (Hatten, J. Cell Biol., 100, 384-396). The cerebellar granule cells were plated in chemically defined neurobasal medium supplemented with B27 and 0.2 mM glutamine, 100 U/ml penicillin, and 0.1 mg/ml streptomycin (Life Technologies). To assess the neutralization of inhibitory acitivity, substrate-coated wells were first incubated with 100 μg/ml of the different recombinant F_{ab} fragments dialyzed against NaCl/Pᵢ (137 mM NaCl, 2.7 mM KCl, 1.5 mM KH₂PO₄, 8 mM Na₂HPO₄, pH 7.4) for 20 min at 37° C. The wells were then washed briefly with HBSS and cells were applied in the presence of the F_{ab} fragments.

Assays were stopped after 24 h in culture by adding 4% (w/v) formalin buffered with NaCl/Pᵢ. For assaying the inhibitory substrate properties, the proportion of total cells bearing neurites longer than the diameter of the cell body (indicating that neurite outgrowth was successfully initiated) was determined. Under control conditions, i.e. in the absence of recombinant Nogo-A, 70% of the cerebellar granule neurons formed processes. Quantification of neurite lengths was performed on cultures monitored with a Zeiss Axiophot microscope. Phase contrast pictures were acquired with a 12-bit digital CCD camera (Visicam Visitron, Germany) and analyzed using Metamorph software (Universal Imaging Corporation, West Chester, Pa.). For each well the longest neurites of at least 100 isolated neurons were measured and averaged. Three wells were investigated for each experimental condition.

As shown in FIG. 5, neurite outgrowth of cerebellar granule cells was severely reduced when recombinant Nogo-A was used as a substrate. In contrast, poly-L-lysine promoted extensive attachment of granule cells in its absence as well as robust neurite growth with an average neurite length of approximately 70 μm in 70% of adherent cells. In this in vitro bioassay functional neutralisation of the inhibitory Nogo-A substrate was observed at different degrees for the various engineered F_{ab} fragments (FIG. 5). While the recombinant wild-type IN-1 F_{ab} fragment revealed partial neutralization of Nogo-A activity, as previously demonstrated (Bandtlow et al., 1996 supra), introduction of the mutation Ala^{L32}ØPhe into the V_{L} domain completely abolished this effect. In contrast, the mutants I.2.6(^{L96}V) and, in particular, II.1.8 exhibited significantly stronger neutralizing effects, as revealed by their better fibre growth-promoting activities, even when the concentration of the inhibitory material was raised. None of the applied F_{ab} fragments exerted an effect on neurite outgrowth of cerebellar granule cells under control conditions, i.e. in the absence of Nogo-A. Notably, the stepwise improvement of the biological activity of the mutants I.2.6(^{L96}V) and II.1.8 in comparison with the wild-type IN-1 F_{ab} fragment correlated well with their relative increase in antigen affinity observed in the ELISA experiment (FIG. 2B).

Accordingly, the soluble truncated Nogo-A fragments according to the present invention provide for an assay system which allows identification of substances which neutralize the inhibitory activity of Nogo-A and which thus can be used as diagnostic and therapeutic agent.

1. An isolated truncated Nogo-A polypeptide that corresponds to a truncated form of the Nogo-A protein consisting of the amino acids 174 to 940 of the full length protein of rat Nogo-A (SEQ ID NO: 1, 1163 amino acids) or of the amino acids 246 to 966 of the human full length protein (SEQ ID NO: 2, 1192 amino acids).

2. The polypeptide of claim 1, wherein said truncated form of the Nogo-A protein consists of the amino acids 223 to 940 of the full length protein of rat Nogo-A.

3. The polypeptide of claim 1, wherein said truncated form is a polypeptide that begins with an amino acid residue selected from the amino acids 174 to 233 and that ends at a residue selected from amino acids 890 to 940 of the full length protein of rat Nogo-A.

4. A polypeptide selected from the group consisting of:
a) the polypeptide having the amino acid sequence consisting of amino acid residues 174 to 940 of the full length rat Nogo-A protein (SEQ ID NO: 1);
b) the polypeptide having the amino acid sequence consisting of amino acid residues 233 to 940 of the full length rat Nogo-A protein (SEQ ID NO: 1);
c) the polypeptide having the amino acid sequence consisting of amino acid residues 246 to 966 of the full length human Nogo-A protein (SEQ ID NO:2);
d) a polypeptide having at least 50% sequence identity to any of the polypeptides a) to c) wherein a fragment of the human Nogo-A protein consisting of amino acids 1 to 1024 is excluded; and
e) a fragment of any of the polypeptides a) to d) wherein the fragment consisting of amino acids 624 to 639 of full length rat Nogo-A protein is excluded.

5. A fusion protein consisting of a Nogo-A polypeptide of claim 1 and a fusion partner fused to the N— and/or the C-terminus of the Nogo-A polypeptide.

6. The fusion protein of claim 5, wherein the fusion partner is a protein, a protein domain or a peptide.

7. A nucleic acid molecule encoding a polypeptide of claim 1.

8. The nucleic acid molecule of claim 7 comprising the nucleotide sequence selected from the group consisting of positions 522 to 2822 of the coding sequences of rat Nogo-A deposited under accession number AJ242961 in the EMBL database and of positions 699 to 2822 of the coding sequence of rat Nogo-A deposited under accession number AJ242961 in the EMBL database.

9. A vector comprising a nucleic acid molecule of claim 7.

10. A host cell comprising a vector as defined in claim 9.

11. A method for the production of a Nogo-A polypeptide of claim 1, wherein the Nogo-A polypeptide is produced starting from the nucleic acid coding for the Nogo-A polypeptide by means of an in vitro transcription and translation system and is isolated from this in vitro system or by means of genetic engineering methods in a bacterial or eucaryotic host organism and is isolated from this host organism or its culture.

12. The method of claim 11, wherein the Nogo-A polypeptide is produced by periplasmic expression in a bacterial host.

13. A method for identifying a compound having detectable affinity to a Nogo-A protein, comprising:
(a) contacting a truncated Nogo-A polypeptide as defined in claim 1 with a compound of interest under conditions that allow formation of a complex between the truncated Nogo-A protein and said compound; and
(b) detecting complex formation by means of a suitable signaling method.

14. The method of claim 13, wherein the compound of interest protein is an organic molecule, a peptide, a polypeptide or a nucleic acid.

15. The method of claim 14, wherein the polypeptide, the peptide or the nucleic acid is subjected to mutagenesis before contacting it with said truncated Nogo-A protein in step a).

16. The method of claim 13, wherein the polypeptide is selected from the group consisting of antibodies and muteins based on a polypeptide of the lipocalin family.

17. The method of claim 16, wherein the antibody is a mutein derived from the antibody IN-1 or a fragment or fusion protein thereof.

18. The method of claim 13, wherein the compound having binding affinity to a Nogo-A protein has a neutralizing effect on the neurite-growth-inhibiting activity of Nogo-A.

19. A method for identifying a compound having detectable affinity to a Nogo-A protein comprising the steps of:
(a) contacting a truncated Nogo-A polypeptide as defined in claim 1 with a plurality of compounds of interest under conditions that allow formation of a complex between the truncated Nogo-A protein and said compounds; and
(b) enriching at least one compound of interest that has detectable binding affinity to the Nogo-A protein by screening or selection and/or isolating said at least one compound.

20. The method of claim 19, wherein the plurality of compounds of interest are selected from the group consisting of peptides, a polypeptides and nucleic acids that have been subjected to mutagenesis before contacting it with said truncated Nogo-A protein in step a).

21. An antibody or an fragment thereof having the variable domains of SEQ ID NO: 11 and SEQ ID NO: 12.
